(19) United States
(12) Patent Application Publication
(10) Pub. No.: US 2026/0283664 A1
(43) Pub. Date: Sep. 24, 2026

(54) METHOD AND APPARATUS FOR AN ORTHOPEDIC FIXATION SYSTEM

(71) Applicant: DePuy Synthes Products, Inc., Raynham, MA (US)

(72) Inventor: Kevin Clancy, West Chester, PA (US)

(73) Assignee: DePuy Synthes Products, Inc., Raynham, MA (US)

(21) Appl. No.: 19/675,936

(22) Filed: May 13, 2026

Related U.S. Application Data
(62) Division of application No. 19/017,167, filed on Jan. 10, 2025.

(51) Int. Cl. A61B 17/86; A61B 17/00; A61B 17/88

(52) U.S. Cl. CPC A61B 17/863; A61B 17/864; A61B 17/866; A61B 17/8888; A61B 2017/00862; A61B 2017/00867

(57) ABSTRACT

An orthopedic fixation system includes a fastener configured to secure a first bone with a second bone whereby the fastener stabilizes the first bone relative to the second bone. The fastener includes an elastic component that provides the fastener with the ability to accommodate relative motion between the first bone relative to the second bone.

### BACKGROUND OF THE INVENTION

### 1. Field of the Invention

The present invention relates generally to an orthopedic fixation system for affixing bone, bones, or bone pieces and, more particularly, but not way of limitation, to an orthopedic fastener configured to affix a first bone and a second bone, the fastener including an elastic component that provides the fastener with the ability to stabilize the first bone relative to the second bone while accommodating relative motion therebetween.

### 2. Description of the Related Art

Syndesmosis is a joint, typically a movable joint formed by a ligament, between a first bone and a second bone that allows relative motion between the first bone and the second bone. An example syndesmosis includes the fibula and the tibia and the articulation thereof at the ankle. A syndesmosis injury requiring syndesmosis repair including bone fixation occurs when an overload of force delivered at the joint between the first bone (e.g., fibula) and the second bone (e.g., tibia) results in a fracture of the first bone, the second bone, or both the first and second bones.

A traditional syndesmosis repair involves a rigid fixation of the syndesmosis at the first bone and the second bone using one or more screws secured with the first and/or second bones and/or one or more bone plates secured by screws with the first and/or second bones. While the rigid fixation initially promotes syndesmosis healing through stabilization of the first bone relative to the second bone, the rigid fixation restricts the normal relative motion between the first bone and the second bone, thereby subjecting the rigid fixation to a high cycle fatigue that often produces broken screws. In accordance therewith, screw fixation for syndesmosis repair often is too rigid resulting in a race between syndesmosis healing and an ultimate screw breakage that precipitates an inadequate syndesmosis repair.

An alternative syndesmosis repair involves a suture-based fixation of the syndesmosis at the first bone and the second bone wherein sutures inserted through the first and second bones are held in place by a plate pair with each plate placed atop the first and second bones such that sutures fixate the first bone with the second bone. While the suture-based fixation initially promotes syndesmosis healing through stabilization of the first bone relative to the second bone, the suture-based fixation, the reliability of which depends upon the tensioning of the suture connections, tends to lax over time, particularly when the plates, due to a misalignment of the tensioned sutures, respectively rub against the first and second bones, thereby tunneling out the first and second bones. In accordance therewith, a suture-based fixation for syndesmosis repair often loses tension too rapidly precipitating an inadequate syndesmosis repair.

Accordingly, an orthopedic fixation system including a fastener with an elastic component whereby the fastener fixates a first bone relative to a second bone while accommodating motion therebetween will provide an improvement in syndesmosis repair.

### SUMMARY OF THE INVENTION

In accordance with the present invention, an orthopedic fixation system includes a fastener configured to secure a first bone with a second bone whereby the fastener stabilizes the first bone relative to the second bone. The fastener includes an elastic component that provides the fastener with the ability to accommodate relative motion between the first bone relative to the second bone. The elastic component allows the fastener to move from a natural insertion shape to an elastically deformed shape in response to a stress applied to the fastener during a relative motion between the first bone and the second bone. The elastic component further allows the fastener to return from the elastically deformed shape to the natural insertion shape upon a removal of the stress applied to the fastener. The elastic component accordingly provides the fastener with the ability to repeatedly cycle between the natural insertion shape and the elastically deformed shape whereby the fastener accommodates relative motion between the first bone relative to the second bone.

The fastener comprises a screw configured to secure a first bone with a second bone whereby the screw stabilizes the first bone relative to the second bone. The screw includes an upper shaft with a top end and a bottom end and threads about at least a segment of the upper shaft and a lower shaft with a top end and a bottom end and threads about at least a segment of the lower shaft. The screw further includes an elastic member connecting the upper shaft at the bottom end thereof with the lower shaft at the top end thereof such that the elastic member provides the screw with the ability to accommodate relative motion between the first bone and the second bone. The elastic member allows the screw to move from a natural insertion shape to an elastically deformed shape in response to a stress applied to the screw during a relative motion between the first bone and the second bone. The elastic member further allows the screw to return from the elastically deformed shape to the natural insertion shape upon a removal of the stress applied to the screw. The elastic member accordingly provides the screw with the ability to repeatedly cycle between the natural insertion shape and the elastically deformed shape whereby the screw accommodates relative motion between the first bone relative to the second bone.

The elastic member comprises a shape memory material that provides the screw with the ability to repeatedly cycle between the natural insertion shape and the elastically deformed shape whereby the screw accommodates relative motion between the first bone relative to the second bone. More particularly, the elastic member comprises a spring formed from a superelastic or elastic shape memory material. The spring connects the upper shaft at the bottom end thereof with the lower shaft at the top end thereof whereby the spring provides the screw with the ability to repeatedly cycle between the natural insertion shape and the elastically deformed shape such that the screw accommodates relative motion between the first bone relative to the second bone. The screw in the natural insertion shape is configured to insert at the lower shaft through the first bone and into the second bone and at the upper shaft into the first bone, whereby the lower shaft inserts into the second bone and the upper shaft inserts into the first bone until the elastic member in the form of the spring resides between the first bone and the second bone.

The screw includes a first drive at the top end of the upper shaft and a second drive at the top end of the lower shaft. The screw further includes a passage from the top end to the bottom end of the upper shaft and a passage through the elastic member communicating with the passage of the upper shaft while being open to the drive at the top end of the lower shaft. The screw still further includes a passage from the top end to the bottom end of the lower shaft. The passage through the elastic member communicates with the passage of the lower shaft to allow the screw at the upper shaft, the elastic member, and the lower shaft to insert over a guide wire utilized to hold the first bone and the second bone during insertion of the screw.

The orthopedic fixation system includes a driver instrument. The driver instrument includes an upper shaft having a top end and a bottom end and a first driver at the bottom end of the upper shaft. The driver instrument includes a lower shaft having a top end and a bottom end and a second driver at the bottom end of the lower shaft. The lower shaft extends at the top end thereof from the bottom end of the upper shaft. The driver instrument is configured to pass through the passages of the upper shaft and the elastic member of the screw whereby the second driver engages the second drive concurrent with the first driver engaging the first drive. The driver instrument further includes a passage from the top end of the upper shaft to the bottom end of the lower shaft to allow insertion of the driver instrument over a guide wire utilized to hold the first bone and the second bone during insertion of the screw.

The driver instrument upon concurrent engagement of the second driver with the second drive and the first driver with the first drive holds the screw in the natural insertion shape. In accordance therewith, the screw in the natural insertion shape inserts using the driver instrument at the lower shaft through the first bone and into the second bone and at the upper shaft into the first bone using the external driver adapted to engage the drive at the top end of the lower shaft concurrently with the drive at the top end of the upper shaft, whereby the lower shaft inserts into the second bone and the upper shaft inserts into the first bone until the elastic member resides between the first bone and the second bone.

The elastic member comprises a series of interconnected individual geometric shapes formed from a superelastic shape memory material. The series of interconnected individual geometric shapes connects the upper shaft at the bottom end thereof with the lower shaft at the top end thereof whereby the series of interconnected individual geometric shapes provides the screw with the ability to repeatedly cycle between the natural insertion shape and the elastically deformed shape such that the screw accommodates relative motion between the first bone relative to the second bone. The screw in the natural insertion shape is configured to insert at the lower shaft through the first bone and into the second bone and at the upper shaft into the first bone, whereby the lower shaft inserts into the second bone and the upper shaft inserts into the first bone until the series of interconnected individual geometric shapes resides between the first bone and the second bone.

The screw in the upper shaft includes a bore from the bottom end to within the upper shaft. Similarly, the screw in the lower shaft includes a bore from the top end to within the lower shaft. The elastic member comprises an elastomer connecting the upper shaft at the bottom end thereof with the lower shaft at the top end thereof whereby the elastomer provides the screw with the ability to repeatedly cycle between the natural insertion shape and the elastically deformed shape such that the screw accommodates relative motion between the first bone relative to the second bone. More particularly, the elastic member comprises an elastomer formed into a bridge with an upper rod extending from the bridge at the top end thereof to within the bore of the upper shaft and a lower rod extending from the bridge at the bottom end thereof to within the bore of the lower shaft such that the bridge at the upper rod and the lower rod connects the upper shaft at the bottom end thereof with the lower shaft at the top end thereof while residing therebetween. The screw in the natural insertion shape being is to insert at the lower shaft through the first bone and into the second bone and at the upper shaft into the first bone, whereby the lower shaft inserts into the second bone and the upper shaft inserts into the first bone until the bridge resides between the first bone and the second bone.

The screw in the upper shaft includes at least one fin extending from the bottom end of the upper shaft. The screw further in the lower shaft includes at least one cavity from the top end to within the lower shaft. The fin and the cavity are configured whereby a positioning of the upper shaft at the bottom end adjacent the lower shaft at the top end and an insertion of the fin into the cavity creates a channel running between the bottom end of the upper shaft and the top end of the lower shaft and the fin and the cavity. The elastic member comprises an elastomer filling disposed within the channel to form a bridge between the bottom end of the upper shaft and the top end of the lower shaft and the fin and the cavity such that the bridge connects the upper shaft at the bottom end thereof with the lower shaft at the top end thereof. The screw in the natural insertion shape is configured to insert at the lower shaft through the first bone and into the second bone and at the upper shaft into the first bone, whereby the lower shaft inserts into the second bone and the upper shaft inserts into the first bone until the bridge resides between the first bone and the second bone.

It is therefore an object of the present invention to provide an orthopedic fixation system with a fastener configured to secure a first bone with a second bone whereby the fastener stabilizes the first bone relative to the second bone.

It is another object of the present invention to provide an orthopedic fixation system with a fastener including an elastic component that provides the fastener with the ability to accommodate relative motion between the first bone relative to the second bone.

It is a further object of the present invention to provide an orthopedic fixation system with a fastener including an elastic component that provides the fastener with the ability to repeatedly cycle between a natural insertion shape and an elastically deformed shape.

Still other objects, features, and advantages of the present invention will become evident to those of ordinary skill in the art in light of the following. Also, it should be understood that the scope of this invention is intended to be broad, and any combination of any subset of the features, elements, or steps described herein is part of the intended scope of the invention.

### BRIEF DESCRIPTION OF DRAWINGS

FIG. 1A is an isometric view illustrating a fastener of an orthopedic fixation system according to a first embodiment.

FIG. 1B is a side view illustrating the fastener of the orthopedic fixation system according to the first embodiment.

FIG. 1C is a top end view illustrating the fastener of the orthopedic fixation system according to the first embodiment.

FIG. 1D is a side view in cross-section taken along line A-A of FIG. 1B illustrating the fastener of the orthopedic fixation system according to the first embodiment.

FIG. 1E is a top end view in cross-section taken along line B-B of FIG. 1B illustrating the fastener of the orthopedic fixation system according to the first embodiment.

FIG. 2A is an isometric view illustrating a fastener of an orthopedic fixation system according to a second embodiment.

FIG. 2B is a side view illustrating the fastener of the orthopedic fixation system according to the second embodiment.

FIG. 2C is a top end view illustrating the fastener of the orthopedic fixation system according to the second embodiment.

FIG. 2D is a side view in cross-section taken along line C-C of FIG. 2B illustrating the fastener of the orthopedic fixation system according to the second embodiment.

FIG. 2E is a top end view in cross-section taken along line D-D of FIG. 2B illustrating the fastener of the orthopedic fixation system according to the first embodiment.

FIG. 3A is an isometric view illustrating a fastener of an orthopedic fixation system according to a third embodiment.

FIG. 3B is a side view illustrating the fastener of the orthopedic fixation system according to the third embodiment.

FIG. 3C is a top end view illustrating the fastener of the orthopedic fixation system according to the third embodiment.

FIG. 3D is a side view in cross-section taken along line E-E of FIG. 3B illustrating the fastener of the orthopedic fixation system according to the third embodiment.

FIG. 3E is a top end view in cross-section taken along line F-F of FIG. 3B illustrating the fastener of the orthopedic fixation system according to the third embodiment.

FIG. 4A is an isometric view illustrating a fastener of an orthopedic fixation system according to a fourth embodiment.

FIG. 4B is a side view illustrating the fastener of the orthopedic fixation system according to the fourth embodiment.

FIG. 4C is a top end view illustrating the fastener of the orthopedic fixation system according to the fourth embodiment.

FIG. 5A is an isometric view illustrating a fastener of an orthopedic fixation system according to a fifth embodiment.

FIG. 5B is a side view illustrating the fastener of the orthopedic fixation system according to the fifth embodiment.

FIG. 5C is a side view in cross-section taken along line G-G of FIG. 5B illustrating the fastener of the orthopedic fixation system according to the fifth embodiment.

FIG. 6A is a side view illustrating an upper shaft of the fastener of the orthopedic fixation system according to the fifth embodiment.

FIG. 6B is a bottom end view illustrating the upper shaft of the fastener of the orthopedic fixation system according to the fifth embodiment.

FIG. 6C is a top end view illustrating the upper shaft of the fastener of the orthopedic fixation system according to the fifth embodiment.

FIG. 6D is a side view in cross-section taken along line H-H of FIG. 6A illustrating the upper shaft of the fastener of the orthopedic fixation system according to the fifth embodiment.

FIG. 7A is a side view illustrating a lower shaft of the fastener of the orthopedic fixation system according to the fifth embodiment.

FIG. 7B is a top end view illustrating the lower shaft of the fastener of the orthopedic fixation system according to the fifth embodiment.

FIG. 7C is a side view in cross-section taken along line I-I of FIG. 7C illustrating the lower shaft of the fastener of the orthopedic fixation system according to the fifth embodiment.

FIG. 8A is an isometric view illustrating a fastener of an orthopedic fixation system according to a sixth embodiment.

FIG. 8B is a side view illustrating the fastener of the orthopedic fixation system according to the sixth embodiment.

FIG. 8C is a side view in cross-section taken along line J-J of FIG. 8B illustrating the fastener of the orthopedic fixation system according to the sixth embodiment.

FIG. 9A is a side view illustrating an upper shaft of the fastener of the orthopedic fixation system according to the sixth embodiment.

FIG. 9B is a bottom end view illustrating the upper shaft of the fastener of the orthopedic fixation system according to the sixth embodiment.

FIG. 9C is a top end view illustrating the upper shaft of the fastener of the orthopedic fixation system according to the sixth embodiment.

FIG. 9D is a side view in cross-section taken along line K-K of FIG. 9A illustrating the upper shaft of the fastener of the orthopedic fixation system according to the sixth embodiment.

FIG. 10A is a side view illustrating a lower shaft of the fastener of the orthopedic fixation system according to the sixth embodiment.

FIG. 10B is a top end view illustrating the lower shaft of the fastener of the orthopedic fixation system according to the sixth embodiment.

FIG. 10C is a side view in cross-section taken along line L-L of FIG. 10C illustrating the lower shaft of the fastener of the orthopedic fixation system according to the sixth embodiment.

FIG. 11A is an isometric view illustrating a fastener of an orthopedic fixation system according to a seventh embodiment.

FIG. 11B is a side view illustrating the fastener of the orthopedic fixation system according to the seventh embodiment.

FIG. 11C is a side view in cross-section taken along line M-M of FIG. 11B illustrating the fastener of the orthopedic fixation system according to the seventh embodiment.

FIG. 12A is an isometric view illustrating an upper shaft of the fastener of the orthopedic fixation system according to the seventh embodiment.

FIG. 12B is a side view illustrating the upper shaft of the fastener of the orthopedic fixation system according to the seventh embodiment.

FIG. 12C is a bottom end view illustrating the upper shaft of the fastener of the orthopedic fixation system according to the seventh embodiment.

FIG. 13A is an isometric view illustrating a lower shaft of the fastener of the orthopedic fixation system according to the seventh embodiment.

FIG. 13B is a side view illustrating the lower shaft of the fastener of the orthopedic fixation system according to the seventh embodiment.

FIG. 13C is a top end view illustrating the lower shaft of the fastener of the orthopedic fixation system according to the seventh embodiment.

FIG. 13D is a side view in cross-section taken along line N-N of FIG. 13A illustrating the lower shaft of the fastener of the orthopedic fixation system according to the seventh embodiment.

FIG. 14A is an isometric view illustrating a fastener of an orthopedic fixation system according to an eighth embodiment.

FIG. 14B is a side view illustrating the fastener of the orthopedic fixation system according to the eighth embodiment.

FIG. 14C is a bottom end view in cross-section taken along line O-O of FIG. 14B illustrating the fastener of the orthopedic fixation system according to the eighth embodiment.

FIG. 14D is a top end view in cross-section taken along line P-P of FIG. 14B illustrating the fastener of the orthopedic fixation system according to the eighth embodiment.

FIG. 15A is an isometric view illustrating a fastener of an orthopedic fixation system according to a ninth embodiment.

FIG. 15B is a side view illustrating the fastener of the orthopedic fixation system according to the ninth embodiment.

FIG. 15C is a side view in cross-section taken along line Q-Q of FIG. 15B illustrating the fastener of the orthopedic fixation system according to the ninth embodiment.

FIG. 15D is a bottom end view in cross-section taken along line R-R of FIG. 15B illustrating the fastener of the orthopedic fixation system according to the eighth embodiment.

FIG. 15E is a top end view in cross-section taken along line S-S of FIG. 15B illustrating the fastener of the orthopedic fixation system according to the ninth embodiment.

FIG. 16A is an isometric view illustrating a driver instrument of the orthopedic fixation system.

FIG. 16B is a side view illustrating the driver instrument of the orthopedic fixation.

FIG. 17 is an isometric view illustrating implantation of the fastener according to the first embodiment into a first bone and a second bone.

FIG. 18 is an isometric view illustrating implantation of multiple fasteners according to the first embodiment into a first bone and a second bone.

FIG. 19 is an isometric view illustrating implantation of the fastener according to the fourth embodiment into a first bone and a second bone.

FIGS. 20A-20B are isometric views illustrating implantation of the fastener according to the first embodiment into a first bone and a second bone in combination with use of a bone plate.

### DETAILED DESCRIPTION OF THE PREFERRED EMBODIMENTS

As required, detailed embodiments of the present invention are disclosed herein; however, it is to be understood that the disclosed embodiments are merely exemplary of the invention, which may be embodied in various forms. Figures are not necessarily to scale, and some features may be exaggerated to show details of particular components or steps.

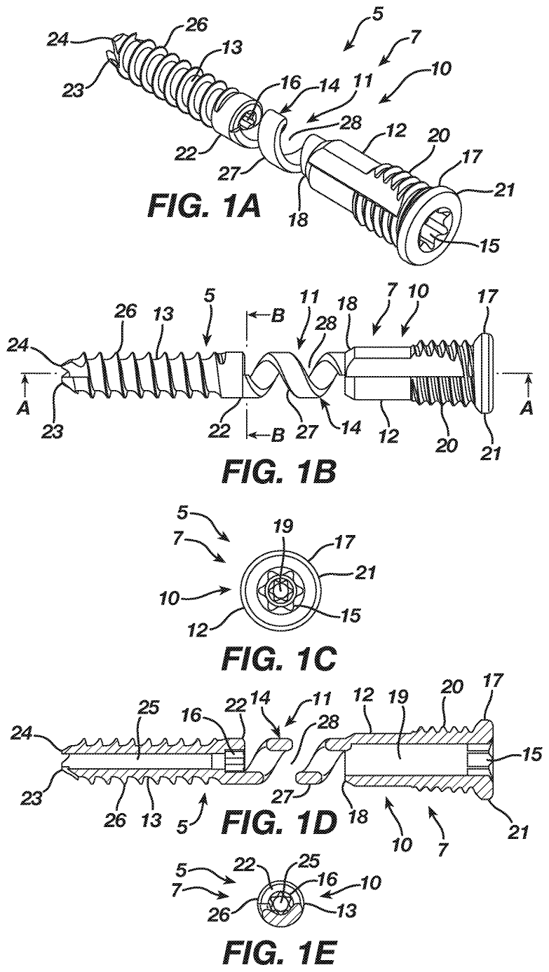

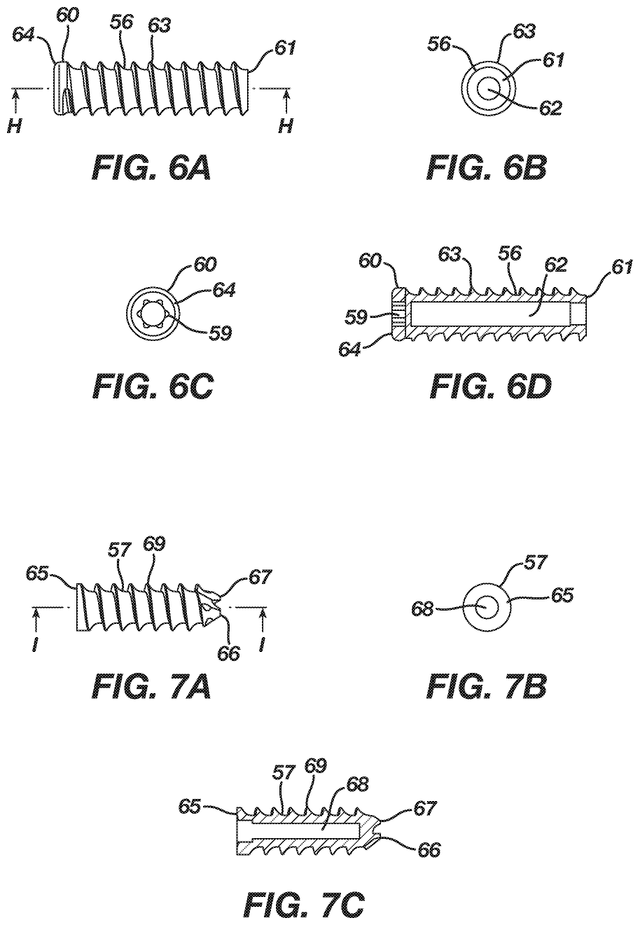

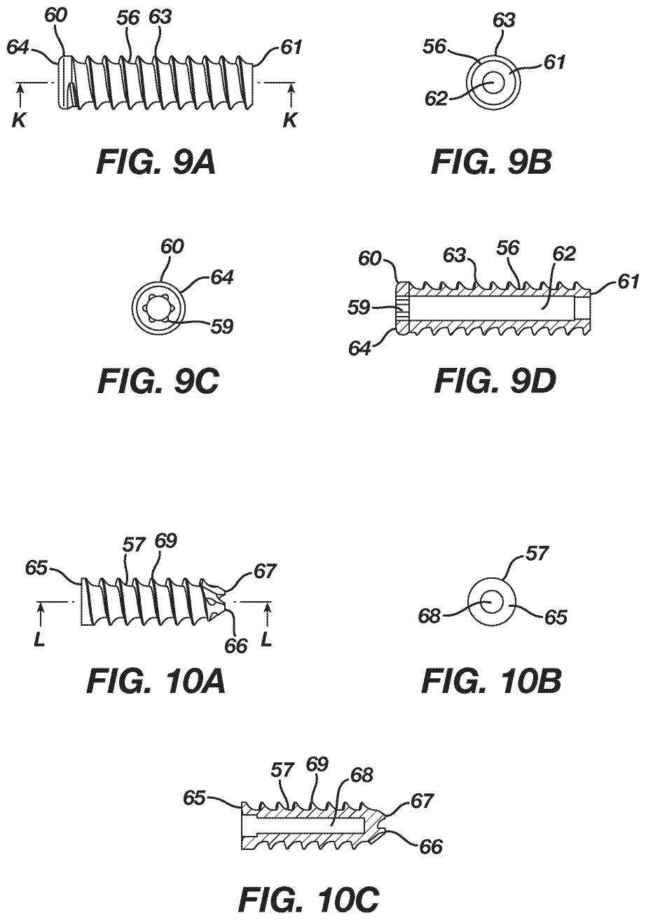

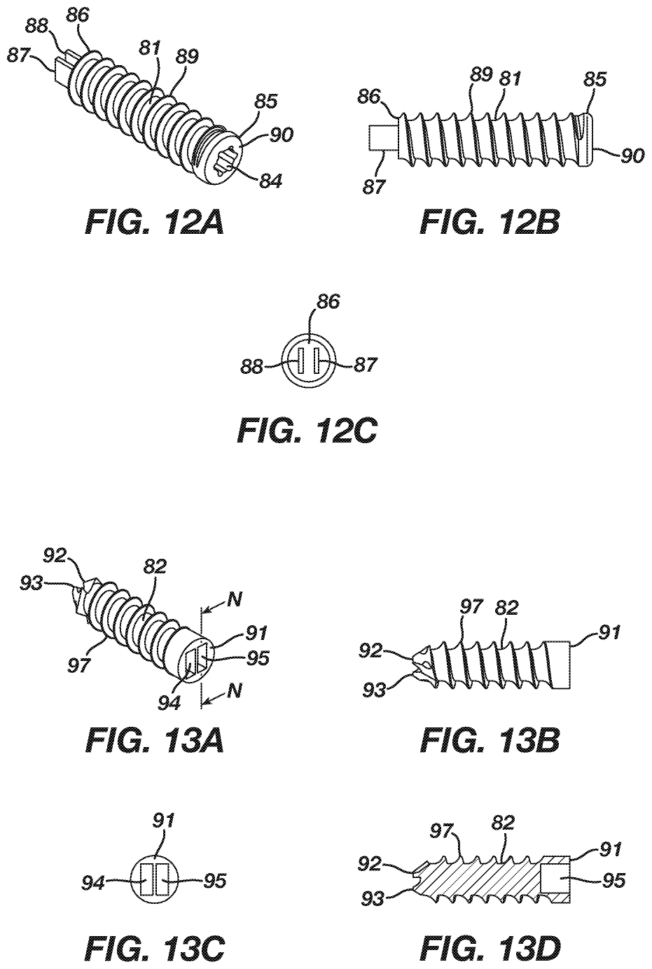

As illustrated in FIGS. 1A-20B with specific reference to FIGS. 1A-15E, an orthopedic fixation system 5 according to the following preferred embodiments includes a fastener 7 in the form of a screw. The fastener 7 in the form of a screw is configured to affix bone, bones, or bone pieces and, more particularly, as presented herein as an example, a first bone and a second bone. The first bone and the second bone in the preferred embodiments include but are not limited to individual bones or a bone with a fracture dividing the bone into two or more pieces. The fastener 7 in the form of a screw in the preferred embodiments includes an elastic component 11 that provides the fastener 7 and thus the screw with the ability to stabilize the first bone relative to the second bone while accommodating relative motion therebetween. Relative motion between the first bone and the second bone includes but is not limited to translational motion and rotational motion. The fastener 7 and thus the screw, due to the inclusion of the elastic component 11, is configured to flex, elongate, and rotate and thus accommodate translational and rotational motion between the first bone and the second bone thereby providing an improvement in bone repair and in particular a syndesmosis repair involving the first and second bones.

FIGS. 1A-1E illustrate the orthopedic fixation system 5 including the fastener 7 in the form of a screw 10 according to a first embodiment. The screw 10 in the first embodiment includes an upper shaft 12, a lower shaft 13, an elastic member 14 providing the elastic component 11, a first drive 15, and a second drive 16. The upper shaft 12 includes a top end 17, a bottom end 18, and a passage 19 from the top end 17 to the bottom end 18. The upper shaft 12 includes threads 20 about the upper shaft 12 or along at least a segment thereof. The upper shaft 12 at the top end 17 in the first embodiment includes a head 21 of the screw 10. The lower shaft 13 includes a top end 22, a bottom end 23 terminating in a point 24 of the screw 10, and a passage 25 from the top end 22 to the bottom end 23 and through the point 24. The lower shaft includes threads 26 about the lower shaft 13 or along at least a segment thereof.

The first drive 15 resides at the top end 17 of the upper shaft 12 and more particularly within the head 21 of the screw 10. The first drive 15 in the first embodiment is any internal drive, such as the illustrated internal hexalobular drive often referred to as a star or torx drive, suitable for engagement by an external driver to facilitate a turning of the screw 10 at the upper shaft 12. The first drive 15 communicates with the passage 19 to allow an external driver as will be described more fully herein to pass through the upper shaft 12 and engage the second drive 16. The first drive 15 and the passage 19 further allow the screw 10 at the upper shaft 12 to insert over a guide wire utilized to hold the first bone and the second bone during insertion of the screw 10.

The second drive 16 resides at the top end 22 of the lower shaft 13. The second drive 16 in the first embodiment is any internal drive, such as the illustrated internal hexalobular drive often referred to as a star or torx drive, suitable for engagement by an external driver to facilitate a turning of the screw 10 at the lower shaft 13. The second drive 16 communicates with the passage 25 to allow the screw 10 at the lower shaft 13 to insert over a guide wire utilized to hold the first bone and the second bone during insertion of the screw 10.

The elastic member 14 forming the elastic component 11 of the screw 10 connects the upper shaft 12 at the bottom end 18 thereof with the lower shaft 13 at the top end 22 thereof. The elastic member 14 provides the screw 10 with the ability to accommodate relative motion between the first bone and the second bone. The elastic member 14 in the first embodiment includes a helical spring 27 comprised of a biocompatible shape memory material exhibiting superelastic properties (e.g., Nitinol). The elastic member 14, due to the helical spring 27, includes a passage 28 therethrough. The passage 28 communicates with the passage 19 of the upper shaft 12 at the bottom end 18 thereof while being open to the second drive 16 at the top end 22 of the lower shaft 13 to allow an external driver to pass through the upper shaft 12 and the elastic member 14 and engage the second drive 16. The passage 28 allows the screw 10 to insert over a guide wire utilized to hold the first bone and the second bone during insertion of the screw 10.

The screw 10 including the elastic member 14 may be formed as one piece from the biocompatible shape memory material using suitable means, such as, for example, machining. Alternatively, the upper and lower shafts 12 and 13 including the first and second drives 15 and 16 of the screw 10 may be formed from a biocompatible metal or metal alloy, such as, for example, titanium, stainless steel, titanium alloy, and cobalt chrome alloy, using suitable means, such as, for example, machining, while the elastic member 14 in the form of the helical spring 27 is formed from the biocompatible shape memory material using suitable means, such as, for example, machining. The elastic member 14 in the form of the helical spring 27 may be secured between the upper shaft 12 at the bottom end 18 thereof and the lower shaft 13 at the top end 22 thereof using suitable means, such as, for example, brazing or soldering.

The screw 10 as illustrated in FIGS. 1A-1E includes a natural insertion shape. Nevertheless, the screw 10 at the elastic member 14, due to the manufacture of the elastic member 14 in the form of the helical spring 27 from a shape memory material, is elastically deformable. In accordance therewith, the elastic member 14 and thus the screw 10 move from the natural insertion shape into an elastically deformed shape responsive to an applied force exerting stress on the screw 10. More particularly, the elastic member 14 and thus the screw 10 flex, elongate, and/or rotate during transition to the elastically deformed shape in response to an applied force exerting stress on the screw 10. The elastic member 14 and thus the screw 10 remain in the elastically deformed shape until removal of the applied force and the resulting stress exerted on the screw 10, whereupon, the elastic member 14 and thus the screw 10, due to the manufacture of the elastic member 14 in the form of the helical spring 27 from the shape memory material, returns from the elastically deformed shape to the natural insertion shape. The elastic member 14 in the form of the helical spring 27 and thus the screw 10 on account of the superelastic properties of the shape memory material is capable of repeated cycling between the natural insertion shape and an elastically deformed shape without experiencing a high cycle fatigue resulting in a breaking of the screw 10.

A bone repair involving the first bone and the second bone, such as, for example, a syndesmosis repair, includes using the screw 10 to affix the first bone and the second bone at, for example, the syndesmosis thereof. The screw 10, which resides in the natural insertion shape, inserts at the lower shaft 13 through the first bone and into the second bone and at the upper shaft 12 into the first bone. The lower shaft 13 inserts into the second bone and the upper shaft 12 inserts into the first bone until the elastic member 14 and more particularly the helical spring 27 resides between and thus spans the first bone and the second bone. In accordance with the lower shaft 13 and the upper shaft 12 inserting respectively into the second bone and the first bone with the elastic member 14 therebetween, the screw 10 fixates and thus stabilizes the first bone relative to the second bone while the elastic member 14 in the form of the helical spring 27 accommodates relative motion therebetween. During the healing process, natural patient movement invariably applies a force at the first bone and the second bone resulting in a relative motion therebetween that stresses the screw 10. Responsive to the stress thereon, the screw 10 at the elastic member 14 in the form of the helical spring 27, due to the superelastic properties of the shape memory material, moves, and, in particular, flexes, elongates, and/or rotates, during a transition from the natural insertion shape to the elastically deformed shape. The screw 10 at the elastic member 14 remains elastically deformed until a termination of the applied force at the first bone and the second bone and the resulting stress on the screw 10. The screw 10 at the elastic member 14, due to the superelastic properties of the shape memory material and in response to the termination of the stress thereon, returns from the elastically deformed shape to the natural insertion shape. Although natural patient movement during the healing process repeatedly stresses the screw 10, the screw 10 manages the recurring stress without breaking on the basis the elastic member 14 in the form of the helical spring 27 and thus the screw 10 on account of the superelastic properties of the shape memory material is capable of repeated cycling between the natural insertion shape and the elastically deformed shape without experiencing a high cycle fatigue resulting in a breaking of the screw 10. The screw 10 therefore provides an improvement in the bone repair of the first bone and the second bone because the screw 10 during the entire healing process fixates and stabilizes the first bone relative to the second bone while the screw 10 at the elastic member 14 in the form of the helical spring 27 accommodates relative motion therebetween, such as, for example, translational and rotational motion.

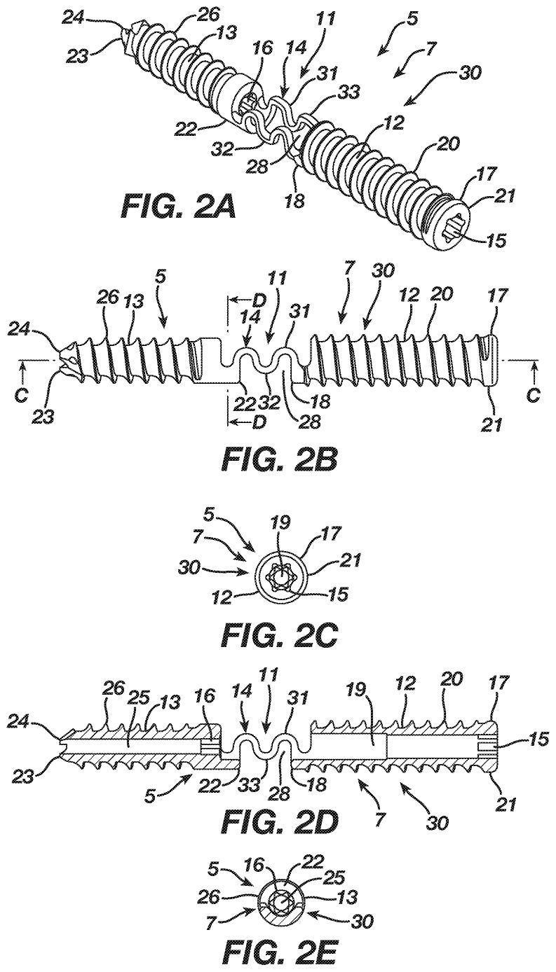

FIGS. 2A-2E illustrate the orthopedic fixation system 5 including the fastener 7 in the form of a screw 30 according to a second embodiment. The screw 30 is substantially similar in design and operation relative to the screw 10 according to the first embodiment such that, for the sake of brevity, only differences therebetween will be described herein. Moreover, one of ordinary skill in the art will recognize that like parts of the screw 30 labeled with like numerals of the screw 10 incorporate a design and function as previously set forth in the detailed description of the screw 10 according to the first embodiment. The screw 10 includes the elastic member 14 in the form of the helical spring 27, whereas the screw 30 includes the elastic member 14 in the form of a linear wave spring 31. The linear wave spring 31 includes at least a first strand 32 and in the second embodiment first and second strands 32 and 33 connecting the upper shaft 12 at the bottom end 18 thereof with the lower shaft 13 at the top end 22 thereof. While the elastic member 14 of the screw 30 is a linear wave spring 31 instead of the helical spring 27, the linear wave spring 31 in the second embodiment comprises a biocompatible shape memory material exhibiting superelastic properties (e.g., Nitinol) whereby the linear wave spring 31 operates substantially, completely identical to the helical spring 27. In accordance therewith, the elastic member 14 in the form of the linear wave spring 31 and thus the screw 30 move from a natural insertion shape illustrated in FIGS. 2A-2E into an elastically deformed shape responsive to an applied force exerting stress on the screw 30. More particularly, the elastic member 14 and thus the screw 30 flex, elongate, and/or rotate during transition to the elastically deformed shape in response to an applied force exerting stress on the screw 30. The elastic member 14 in the form of the linear wave spring 31 and thus the screw 30 on account of the superelastic properties of the shape memory material is capable of repeated cycling between the natural insertion shape and an elastically deformed shape without experiencing a high cycle fatigue resulting in a breaking of the screw 30. The screw 30 therefore provides an improvement in the bone repair of the first bone and the second bone because the screw 30 during the entire healing process fixates and stabilizes the first bone relative to the second bone while the screw 30 at the elastic member 14 in the form of the linear wave spring 31 accommodates relative motion therebetween, such as, for example, translational and rotational motion.

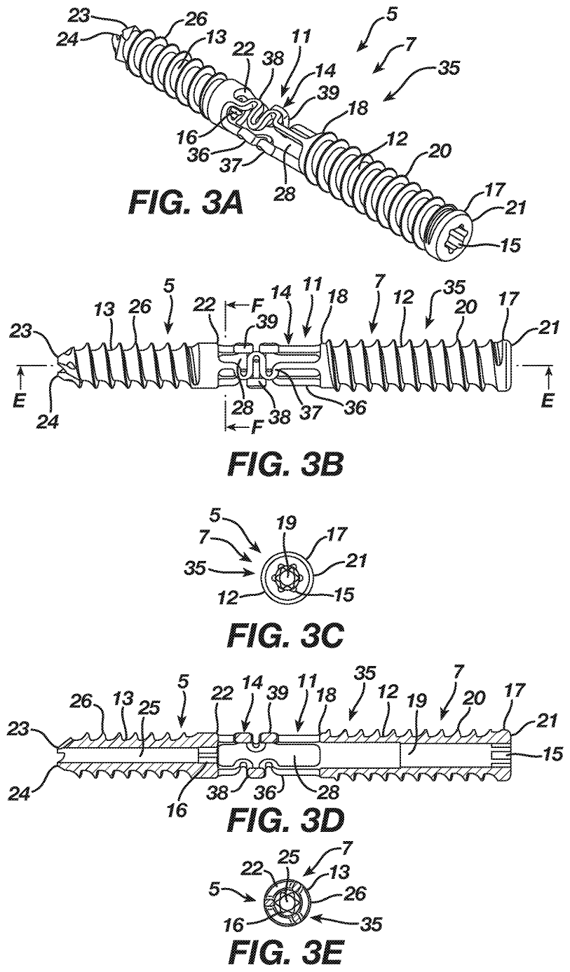

FIGS. 3A-3E illustrate the orthopedic fixation system 5 including the fastener 7 in the form of a screw 35 according to a third embodiment. The screw 35 is substantially similar in design and operation relative to the screw 10 according to the first embodiment such that, for the sake of brevity, only differences therebetween will be described herein. Moreover, one of ordinary skill in the art will recognize that like parts of the screw 35 labeled with like numerals of the screw 10 incorporate a design and function as previously set forth in the detailed description of the screw 10 according to the first embodiment. The screw 10 includes the elastic member 14 in the form of the helical spring 27, whereas the screw 35 includes the elastic member 14 in the form of a linear wave spring 36. The linear wave spring 36 includes at least a first strand 37 and in the third embodiment first, second, and third strands 37, 38, and 39 connecting the upper shaft 12 at the bottom end 18 thereof with the lower shaft 13 at the top end 22 thereof. While the elastic member 14 of the screw 35 is a linear wave spring 36 instead of the helical spring 27, the linear wave spring 36 in the third embodiment comprises a biocompatible shape memory material exhibiting superelastic properties (e.g., Nitinol) whereby the linear wave spring 36 operates substantially, completely identical to the helical spring 27. In accordance therewith, the elastic member 14 in the form of the linear wave spring 36 and thus the screw 35 move from a natural insertion shape illustrated in FIGS. 3A-3E into an elastically deformed shape responsive to an applied force exerting stress on the screw 35. More particularly, the elastic member 14 and thus the screw 35 flex, elongate, and/or rotate during transition to the elastically deformed shape in response to an applied force exerting stress on the screw 35. The elastic member 14 in the form of the linear wave spring 36 and thus the screw 35 on account of the superelastic properties of the shape memory material is capable of repeated cycling between the natural insertion shape and an elastically deformed shape without experiencing a high cycle fatigue resulting in a breaking of the screw 35. The screw 35 therefore provides an improvement in the bone repair of the first bone and the second bone because the screw 35 during the entire healing process fixates and stabilizes the first bone relative to the second bone while the screw 35 at the elastic member 14 in the form of the linear wave spring 36 accommodates relative motion therebetween, such as, for example, translational and rotational motion.

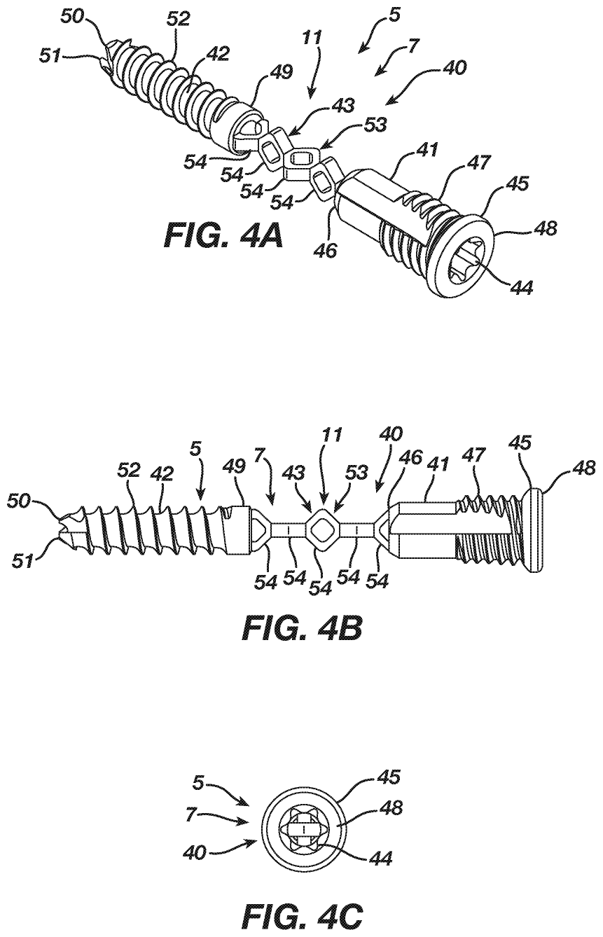

FIGS. 4A-4C illustrate the orthopedic fixation system 5 including the fastener 7 in the form of a screw 40 according to a fourth embodiment. The screw 40 in the fourth embodiment includes an upper shaft 41, a lower shaft 42, an elastic member 43 providing the elastic component 11, and a drive 44. The upper shaft 41 includes a top end 45 and a bottom end 46. The upper shaft 41 includes threads 47 about the upper shaft 41 or along at least a segment thereof. The upper shaft 41 at the top end 45 in the fourth embodiment includes a head 48 of the screw 40. The lower shaft 42 includes a top end 49 and a bottom end 50 terminating in a point 51 of the screw 40. The lower shaft 42 includes threads 52 about the lower shaft 42 or along at least a segment thereof.

The drive 44 resides at the top end 45 of the upper shaft 41 and more particularly within the head 48 of the screw 40. The drive 44 in the fourth embodiment is any internal drive, such as the illustrated internal hexalobular drive often referred to as a star or torx drive, suitable for engagement by an external driver to facilitate a turning of the screw 40 at the upper shaft 41.

The elastic member 43 forming the elastic component 11 of the screw 40 connects the upper shaft 41 at the bottom end 46 thereof with the lower shaft 42 at the top end 49 thereof. The elastic member 43 provides the screw 40 with the ability to accommodate relative motion between the first bone and the second bone. The elastic member 43 in the fourth embodiment includes a series 53 of interconnected individual geometric shapes 54 comprised of a biocompatible shape memory material exhibiting superelastic properties (e.g., Nitinol). The interconnected individual geometric shapes 54 in the fourth embodiment include two or more interconnected sides, such as, for example, the four sided diamond shapes shown in FIGS. 4A and 4B. The interconnected individual geometric shapes 54 in the fourth embodiment reside in alternating planes although the interconnected individual geometric shapes 54 could reside in the same plane or dissimilar planes. The interconnected individual geometric shapes 54 in the fourth embodiment each include openings therethrough although the interconnected individual geometric shapes 54 could be solid. The interconnected individual geometric shapes 54 in the fourth embodiment adjacent the bottom end 46 of the upper shaft 41 and the top end 49 of the lower shaft 42 are truncated to provide multiple engagement points for the elastic member 43 with the upper and lower shafts 41 and 42. The screw 40 including the elastic member 43 may be formed as one piece from the biocompatible shape memory material using suitable means, such as, for example, machining. Alternatively, the upper and lower shafts 41 and 42 including the drive 44 of the screw 40 may be formed from a biocompatible metal or metal alloy, such as, for example, titanium, stainless steel, titanium alloy, and cobalt chrome alloy, using suitable means, such as, for example, machining, while the elastic member 43 in the form of the series 53 of interconnected individual geometric shapes 54 is formed from the biocompatible shape memory material using suitable means, such as, for example, machining. The elastic member 43 in the form of the series 53 of interconnected individual geometric shapes 54 may be secured between the upper shaft 41 at the bottom end 46 thereof and the lower shaft 42 at the top end 49 thereof using suitable means, such as, for example, brazing or soldering.

The screw 40 as illustrated in FIGS. 4A-4C includes a natural insertion shape. Nevertheless, the screw 40 at the elastic member 43, due to the manufacture of the elastic member 43 in the form of the series 53 of interconnected individual geometric shapes 54 from a shape memory material, is elastically deformable. In accordance therewith, the elastic member 43 and thus the screw 40 move from the natural insertion shape into an elastically deformed shape responsive to an applied force exerting stress on the screw 40. More particularly, the elastic member 43 and thus the screw 40 flex, elongate, and/or rotate during transition to the elastically deformed shape in response to an applied force exerting stress on the screw 40. While the elastic member 43 in the form of the series 53 of interconnected individual geometric shapes 54 elastically deforms in response to an applied force exerting stress on the screw 40, the elastic member 43 in the fourth embodiment is sufficiently rigid to permit a rotation of the screw 40 during an insertion thereof into the first bone and the second bone whereby the screw 40 inserts into the first bone and the second bone in the natural insertion shape. If desirable to the operation of the screw 40, the rotation component of the elastic member 43 triggered by a stress on the screw 40 may be eliminated in order to ensure the screw 40 inserts into the first bone and the second bone in the natural insertion shape. The elastic member 43 and thus the screw 40 remain in the elastically deformed shape until removal of the applied force and the resulting stress exerted on the screw 40, whereupon, the elastic member 43 and thus the screw 40, due to the manufacture of the elastic member 43 in the form of the series 53 of interconnected individual geometric shapes 54 from the shape memory material, returns from the elastically deformed shape to the natural insertion shape. The elastic member 43 in the form of the series 53 of interconnected individual geometric shapes 54 and thus the screw 40 on account of the superelastic properties of the shape memory material is capable of repeated cycling between the natural insertion shape and an elastically deformed shape without experiencing a high cycle fatigue resulting in a breaking of the screw 40.

A bone repair involving the first bone and the second bone, such as, for example, a syndesmosis repair, includes using the screw 40 to affix the first bone and the second bone at, for example, the syndesmosis thereof. The screw 40, which resides in the natural insertion shape, inserts at the lower shaft 42 through the first bone and into the second bone and at the upper shaft 41 into the first bone. The lower shaft 42 inserts into the second bone and the upper shaft 41 inserts into the first bone until the elastic member 43 and more particularly the series 53 of interconnected individual geometric shapes 54 resides between and thus spans the first bone and the second bone. In accordance with the lower shaft 42 and the upper shaft 41 inserting respectively into the second bone and the first bone with the elastic member 43 therebetween, the screw 40 fixates and thus stabilizes the first bone relative to the second bone while the elastic member 43 in the form of the series 53 of interconnected individual geometric shapes 54 accommodates relative motion therebetween. During the healing process, natural patient movement invariably applies a force at the first bone and the second bone resulting in a relative motion therebetween that stresses the screw 40. Responsive to the stress thereon, the screw 40 at the elastic member 43 in the form of the series 53 of interconnected individual geometric shapes 54, due to the superelastic properties of the shape memory material, moves, and, in particular, flexes, elongates, and/or rotates, during a transition from the natural insertion shape to the elastically deformed shape. The screw 40 at the elastic member 43 remains elastically deformed until a termination of the applied force at the first bone and the second bone and the resulting stress on the screw 40. The screw 40 at the elastic member 43, due to the superelastic properties of the shape memory material and in response to the termination of the stress thereon, returns from the elastically deformed shape to the natural insertion shape. Although natural patient movement during the healing process repeatedly stresses the screw 40, the screw 40 manages the recurring stress without breaking on the basis the elastic member 43 in the form of the series 53 of interconnected individual geometric shapes 54 and thus the screw 40 on account of the superelastic properties of the shape memory material is capable of repeated cycling between the natural insertion shape and the elastically deformed shape without experiencing a high cycle fatigue resulting in a breaking of the screw 40. The screw 40 therefore provides an improvement in the bone repair of the first bone and the second bone because the screw 40 during the entire healing process fixates and stabilizes the first bone relative to the second bone while the screw 40 at the elastic member 43 in the form of the series 53 of interconnected individual geometric shapes 54 accommodates relative motion therebetween, such as, for example, translational and rotational motion.

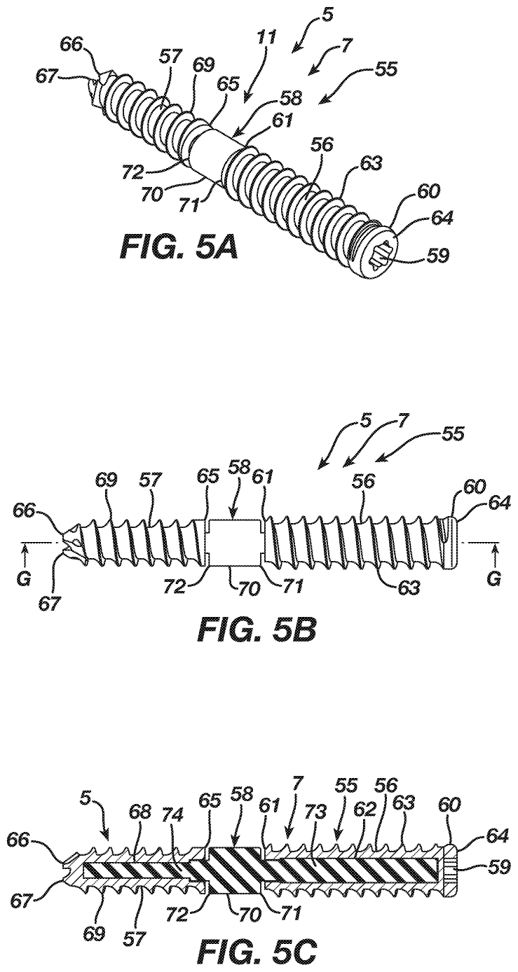

FIGS. 5A-7C illustrate the orthopedic fixation system 5 including the fastener 7 in the form of a screw 55 according to a fifth embodiment. The screw 55 in the fifth embodiment includes an upper shaft 56, a lower shaft 57, an elastic member 58 providing the elastic component 11, and a drive 59. The upper shaft 56 includes a top end 60, a bottom end 61, and a bore 62 from the bottom end 61 to within the upper shaft 56. The upper shaft 56 includes threads 63 about the upper shaft 56 or along at least a segment thereof. The upper shaft 56 at the top end 60 in the fifth embodiment includes a head 64 of the screw 55. The lower shaft 57 includes a top end 65, a bottom end 66 terminating in a point 67 of the screw 55, and a bore 68 from the top end 65 to within the lower shaft 57. The lower shaft 57 includes threads 69 about the lower shaft 57 or along at least a segment thereof.

The drive 59 resides at the top end 60 of the upper shaft 56 and more particularly within the head 64 of the screw 55. The drive 59 in the fifth embodiment is any internal drive, such as the illustrated internal hexalobular drive often referred to as a star or torx drive, suitable for engagement by an external driver to facilitate a turning of the screw 55 at the upper shaft 56.

The elastic member 58 forming the elastic component 11 of the screw 55 connects the upper shaft 56 at the bottom end 61 thereof with the lower shaft 57 at the top end 65 thereof. The elastic member 58 provides the screw 55 with the ability to accommodate relative motion between the first bone and the second bone. The elastic member 58 in the fifth embodiment includes a bridge 70 with a top end 71 and a bottom end 72, an upper rod 73 extending from the bridge 70 at the top end 71, and a lower rod 74 extending from the bridge 70 at the bottom end 72. The elastic member 58 in the fifth embodiment and thus the bridge 70 and the upper and lower rods 73 and 74 comprise a biocompatible shape memory material such as, for example, an elastomer exhibiting elastic properties. The bridge 70 in the fifth embodiment includes any geometric shape suitable to facilitate relative movement between the upper and lower shafts 56 and 57 of the screw 55, such as, for example, the cylinder shown in FIGS. 5A-5C.

The upper and lower shafts 56 and 57 including respectively the bores 62 and 68 therein and the drive 59 of the screw 55 may be formed from a biocompatible metal or metal alloy, such as, for example, titanium, stainless steel, titanium alloy, and cobalt chrome alloy, using suitable means, such as, for example, machining. After forming the upper and lower shafts 56 and 57, an elastomer using any known injection molding technique is introduced between the upper shaft 56 at the bottom end 61 thereof and the lower shaft 57 at the top end 65 thereof and then into the bores 62 and 68 to form the upper and lower rods 73 and 74 respectively within the upper and lower shafts 56 and 57. Upon formation of the upper and lower rods 73 and 74, a continued introduction of the elastomer between the upper shaft 56 at the bottom end 61 thereof and the lower shaft 57 at the top end 65 forms the bridge 70 integrally between the upper and lower rods 73 and 74. The forming of the bridge 70 integrally between the upper and lower rods 73 and 74 with the upper rod 73 extending from the top end 71 of the bridge 70 into the bore 62 of the upper shaft 56 and the lower rod 74 extending from the bottom end 72 of the bridge 70 into the bore 68 of the lower shaft 57 connects the upper shaft 56 with the lower shaft 57 with the bridge 70 between the bottom end 61 of the upper shaft 56 and the top end 65 of the lower shaft 57.

The screw 55 as illustrated in FIGS. 5A-5C includes a natural insertion shape. Nevertheless, the screw 55 at the elastic member 58, due to the manufacture of the elastic member 58 in the form of the bridge 70 having upper and lower rods 73 and 74 from an elastomer, is elastically deformable. In accordance therewith, the elastic member 58 and thus the screw 55 move from the natural insertion shape into an elastically deformed shape responsive to an applied force exerting stress on the screw 55. More particularly, the elastic member 58 and thus the screw 55 flex, elongate, and/or rotate during transition to the elastically deformed shape in response to an applied force exerting stress on the screw 55. While the elastic member 58 in the form of the bridge 70 having upper and lower rods 73 and 74 elastically deforms in response to an applied force exerting stress on the screw 55, the elastic member 58 in the fifth embodiment is sufficiently rigid to permit a rotation of the screw 55 during an insertion thereof into the first bone and the second bone whereby the screw 55 inserts into the first bone and the second bone in the natural insertion shape. If desirable to the operation of the screw 55, the rotation component of the elastic member 58 triggered by a stress on the screw 55 may be eliminated in order to ensure the screw 55 inserts into the first bone and the second bone in the natural insertion shape. The elastic member 58 and thus the screw 55 remain in the elastically deformed shape until removal of the applied force and the resulting stress exerted on the screw 55, whereupon, the elastic member 58 and thus the screw 55, due to the manufacture of the elastic member 58 in the form of the bridge 70 having upper and lower rods 73 and 74 from the elastomer, returns from the elastically deformed shape to the natural insertion shape. The elastic member 58 in the form of the bridge 70 having upper and lower rods 73 and 74 and thus the screw 55 on account of the elastic properties of the elastomer is capable of repeated cycling between the natural insertion shape and an elastically deformed shape without experiencing a high cycle fatigue resulting in a breaking of the screw 55.

A bone repair involving the first bone and the second bone, such as, for example, a syndesmosis repair, includes using the screw 55 to affix the first bone and the second bone at, for example, the syndesmosis thereof. The screw 55, which resides in the natural insertion shape, inserts at the lower shaft 57 through the first bone and into the second bone and at the upper shaft 56 into the first bone. The lower shaft 57 inserts into the second bone and the upper shaft 56 inserts into the first bone until the elastic member 58 and more particularly the bridge 70 resides between and thus spans the first bone and the second bone. In accordance with the lower shaft 57 and the upper shaft 56 inserting respectively into the second bone and the first bone with the elastic member 58 therebetween, the screw 55 fixates and thus stabilizes the first bone relative to the second bone while the elastic member 58 in the form of the bridge 70 having upper and lower rods 73 and 74 accommodates relative motion therebetween. During the healing process, natural patient movement invariably applies a force at the first bone and the second bone resulting in a relative motion therebetween that stresses the screw 55. Responsive to the stress thereon, the screw 55 at the elastic member 58 in the form of the bridge 70 having upper and lower rods 73 and 74, due to the elastic properties of the elastomer, moves, and, in particular, flexes, elongates, and/or rotates, during a transition from the natural insertion shape to the elastically deformed shape. The screw 55 at the elastic member 58 remains elastically deformed until a termination of the applied force at the first bone and the second bone and the resulting stress on the screw 55. The screw 55 at the elastic member 58, due to the elastic properties of the elastomer and in response to the termination of the stress thereon, returns from the elastically deformed shape to the natural insertion shape. Although natural patient movement during the healing process repeatedly stresses the screw 55, the screw 55 manages the recurring stress without breaking on the basis the elastic member 58 in the form of the bridge 70 having upper and lower rods 73 and 74 and thus the screw 55 on account of the elastic properties of the elastomer is capable of repeated cycling between the natural insertion shape and the elastically deformed shape without experiencing a high cycle fatigue resulting in a breaking of the screw 55. The screw 55 therefore provides an improvement in the bone repair of the first bone and the second bone because the screw 55 during the entire healing process fixates and stabilizes the first bone relative to the second bone while the screw 55 at the elastic member 58 in the form of the bridge 70 having upper and lower rods 73 and 74 accommodates relative motion therebetween, such as, for example, translational and rotational motion.

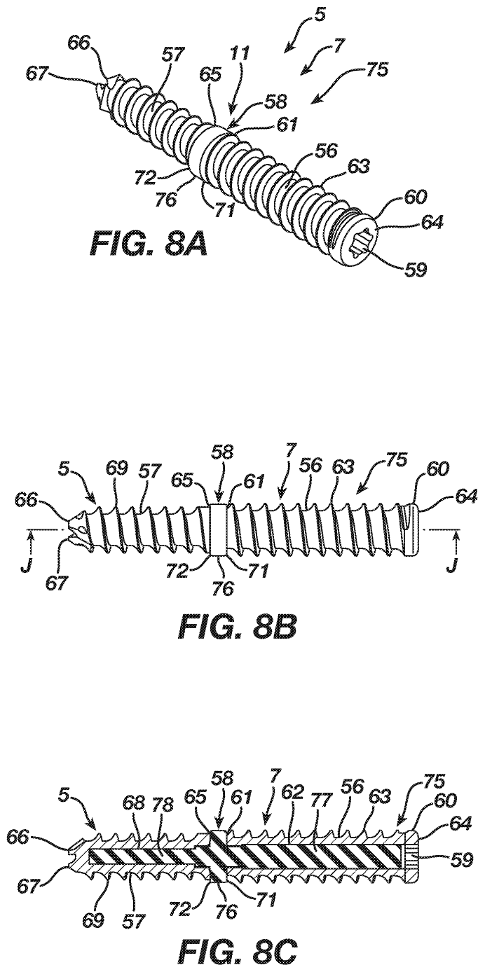

FIGS. 8A-10C illustrate the orthopedic fixation system 5 including the fastener 7 in the form of a screw 75 according to a sixth embodiment. The screw 75 is substantially similar in design and operation relative to the screw 55 according to the fifth embodiment such that, for the sake of brevity, only differences therebetween will be described herein. Moreover, one of ordinary skill in the art will recognize that like parts of the screw 75 labeled with like numerals of the screw 55 incorporate a design and function as previously set forth in the detailed description of the screw 55 according to the fifth embodiment. The screw 55 includes the elastic member 58 in the form of the bridge 70 having the upper and lower rods 73 and 74, whereas the screw 75 includes the elastic member 58 in the form of a bridge 76 having the upper and lower rods 77 and 78. The bridge 76, the upper rod 77, and the lower rod 78 are substantially, completely identical to the bridge 70, the upper rod 73, and the lower rod 74, except the bridge 76 includes a length less than the length of the bridge 70. While the elastic member 58 of the screw 75 includes a bridge 76 with a length less than the length of the bridge 70, the elastic member 58 in the sixth embodiment in the form of the bridge 76 having the upper and lower rods 77 and 78 comprises a biocompatible shape memory material such as, for example, an elastomer exhibiting elastic properties, whereby the elastic member 58 in the form of the bridge 76 having the upper and lower rods 77 and 78 operates substantially, completely identical to the elastic member 58 in the form of the bridge 70 having the upper and lower rods 73 and 74. In accordance therewith, the elastic member 58 in the form of the bridge 76 having the upper and lower rods 77 and 78 and thus the screw 75 move from a natural insertion shape illustrated in FIGS. 8A-8C into an elastically deformed shape responsive to an applied force exerting stress on the screw 75. More particularly, the elastic member 58 and thus the screw 75 flex, elongate, and/or rotate during transition to the elastically deformed shape in response to an applied force exerting stress on the screw 75. While the elastic member 58 in the form of the bridge 76 having the upper and lower rods 77 and 78 elastically deforms in response to an applied force exerting stress on the screw 75, the elastic member 58 in the sixth embodiment is sufficiently rigid to permit a rotation of the screw 75 during an insertion thereof into the first bone and the second bone whereby the screw 75 inserts into the first bone and the second bone in the natural insertion shape. If desirable to the operation of the screw 75, the rotation component of the elastic member 58 triggered by a stress on the screw 75 may be eliminated in order to ensure the screw 75 inserts into the first bone and the second bone in the natural insertion shape. The elastic member 58 in the form of the bridge 76 having the upper and lower rods 77 and 78 and thus the screw 75 on account of the elastic properties of the elastomer is capable of repeated cycling between the natural insertion shape and an elastically deformed shape without experiencing a high cycle fatigue resulting in a breaking of the screw 75. The screw 75 therefore provides an improvement in the bone repair of the first bone and the second bone because the screw 75 during the entire healing process fixates and stabilizes the first bone relative to the second bone while the screw 75 at the elastic member 58 in the form of the bridge 76 having the upper and lower rods 77 and 78 accommodates relative motion therebetween, such as, for example, translational and rotational motion.

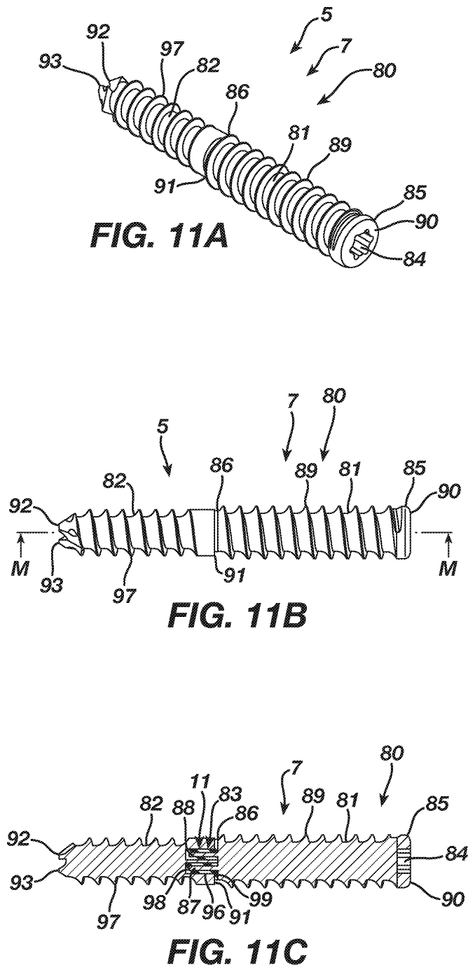

FIGS. 11A-13D illustrate the orthopedic fixation system 5 including the fastener 7 in the form of a screw 80 according to a seventh embodiment. The screw 80 in the seventh embodiment includes an upper shaft 81, a lower shaft 82, an elastic member 83 providing the elastic component 11, and a drive 84. The upper shaft 81 includes a top end 85, a bottom end 86, and at least a first fin 87 extending from the bottom end 86 and in the seventh embodiment first and second fins 87 and 88 extending from the bottom end 86. The upper shaft 81 includes threads 89 about the upper shaft 81 or along at least a segment thereof. The upper shaft 81 at the top end 85 in the seventh embodiment includes a head 90 of the screw 80. The lower shaft 82 includes a top end 91, a bottom end 92 terminating in a point 93 of the screw 80, and at least a first cavity 94 from the top end 91 to within the lower shaft 82 and in the seventh embodiment first and second cavities 94 and 95 from the top end 91 to within the lower shaft 82. The first and second fins 87 and 88 and the first and second cavities 94 and 95 in the seventh embodiment are sized and capable of alignment whereby a positioning of the upper shaft 81 at the bottom end 86 adjacent the lower shaft 82 at the top end 91 and an insertion of the first fin 87 into the first cavity 94 and the second fin 88 into the second cavity 95 creates a channel 96 running between the bottom end 86 of the upper shaft 81 and the top end 91 of the lower shaft 82 and the first and second fins 87 and 88 and the first and second cavities 94 and 95. The lower shaft 82 includes threads 97 about the lower shaft 82 or along at least a segment thereof.

The drive 84 resides at the top end 85 of the upper shaft 81 and more particularly within the head 90 of the screw 80. The drive 84 in the seventh embodiment is any internal drive, such as the illustrated internal hexalobular drive often referred to as a star or torx drive, suitable for engagement by an external driver to facilitate a turning of the screw 80 at the upper shaft 81.

The elastic member 83 forming the elastic component 11 of the screw 80 connects the upper shaft 81 at the bottom end 86 thereof with the lower shaft 82 at the top end 91 thereof. The elastic member 83 provides the screw 80 with the ability to accommodate relative motion between the first bone and the second bone. The elastic member 83 in the seventh embodiment comprises a biocompatible shape memory material such as, for example, an elastomer exhibiting elastic properties. More particularly, as illustrated in FIG. 11C, the elastic member 83 comprises an elastomer filling 98 disposed within the channel 96 forming a bridge 99 between the bottom end 86 of the upper shaft 81 and the top end 91 of the lower shaft 82 and the first and second fins 87 and 88 and the first and second cavities 94 and 95. The bridge 99 in the seventh embodiment accordingly connects the upper shaft 81 at the bottom end 86 thereof with the lower shaft 82 at the top end 91 thereof while facilitating relative movement between the upper and lower shafts 81 and 82 of the screw 80.

The upper and lower shafts 81 and 82 including respectively the first and second fins 87 and 88 and the first and second cavities 94 and 95 and the drive 84 of the screw 80 may be formed from a biocompatible metal or metal alloy, such as, for example, titanium, stainless steel, titanium alloy, and cobalt chrome alloy, using suitable means, such as, for example, machining. After forming the upper and lower shafts 81 and 82, an elastomer using any known injection molding technique is introduced into the channel 96 between the bottom end 86 of the upper shaft 81 and the top end 91 of the lower shaft 82 and the first and second fins 87 and 88 and the first and second cavities 94 and 95 until the elastomer expands into and completely fills the channel 96 thereby creating the elastomer filling 98 and thus the bridge 99 that connects the upper shaft 81 with the lower shaft 82.

The screw 80 as illustrated in FIGS. 11A-11C includes a natural insertion shape. Nevertheless, the screw 80 at the elastic member 83, due to the manufacture of the elastic member 83 in the form of the bridge 99 from the elastomer filling 98, is elastically deformable. In accordance therewith, the elastic member 83 and thus the screw 80 move from the natural insertion shape into an elastically deformed shape responsive to an applied force exerting stress on the screw 80. More particularly, the elastic member 83 and thus the screw 80 flex during transition to the elastically deformed shape in response to an applied force exerting stress on the screw 80. While the elastic member 83 in the form of the bridge 99 elastically deforms in response to an applied force exerting stress on the screw 80, the elastic member 83 in the seventh embodiment is sufficiently rigid to permit a rotation of the screw 80 during an insertion thereof into the first bone and the second bone whereby the screw 80 inserts into the first bone and the second bone in the natural insertion shape. The elastic member 83 and thus the screw 80 remain in the elastically deformed shape until removal of the applied force and the resulting stress exerted on the screw 80, whereupon, the elastic member 83 and thus the screw 80, due to the manufacture of the elastic member 83 in the form of the bridge 99 from the elastomer filling 98, returns from the elastically deformed shape to the natural insertion shape. The elastic member 83 in the form of the bridge 99 and thus the screw 80 on account of the elastic properties of the elastomer filling 98 is capable of repeated cycling between the natural insertion shape and an elastically deformed shape without experiencing a high cycle fatigue resulting in a breaking of the screw 80.

A bone repair involving the first bone and the second bone, such as, for example, a syndesmosis repair, includes using the screw 80 to affix the first bone and the second bone at, for example, the syndesmosis thereof. The screw 80, which resides in the natural insertion shape, inserts at the lower shaft 82 through the first bone and into the second bone and at the upper shaft 81 into the first bone. The lower shaft 82 inserts into the second bone and the upper shaft 81 inserts into the first bone until the elastic member 83 and more particularly the bridge 99 resides between and thus spans the first bone and the second bone. In accordance with the lower shaft 82 and the upper shaft 81 inserting respectively into the second bone and the first bone with the elastic member 83 therebetween, the screw 80 fixates and thus stabilizes the first bone relative to the second bone while the elastic member 83 in the form of the bridge 99 accommodates relative motion therebetween. During the healing process, natural patient movement invariably applies a force at the first bone and the second bone resulting in a relative motion therebetween that stresses the screw 80. Responsive to the stress thereon, the screw 80 at the elastic member 83 in the form of the bridge 99, due to the elastic properties of the elastomer filling 98, moves, and, in particular, flexes during a transition from the natural insertion shape to the elastically deformed shape. The screw 80 at the elastic member 83 remains elastically deformed until a termination of the applied force at the first bone and the second bone and the resulting stress on the screw 80. The screw 80 at the elastic member 83, due to the elastic properties of the elastomer filling 98 and in response to the termination of the stress thereon, returns from the elastically deformed shape to the natural insertion shape. Although natural patient movement during the healing process repeatedly stresses the screw 80, the screw 80 manages the recurring stress without breaking on the basis the elastic member 83 in the form of the bridge 99 and thus the screw 80 on account of the elastic properties of the elastomer filling 98 is capable of repeated cycling between the natural insertion shape and the elastically deformed shape without experiencing a high cycle fatigue resulting in a breaking of the screw 80. The screw 80 therefore provides an improvement in the bone repair of the first bone and the second bone because the screw 80 during the entire healing process fixates and stabilizes the first bone relative to the second bone while the screw 80 at the elastic member 83 in the form of the bridge 99 accommodates relative motion therebetween, such as, for example, translational and rotational motion.

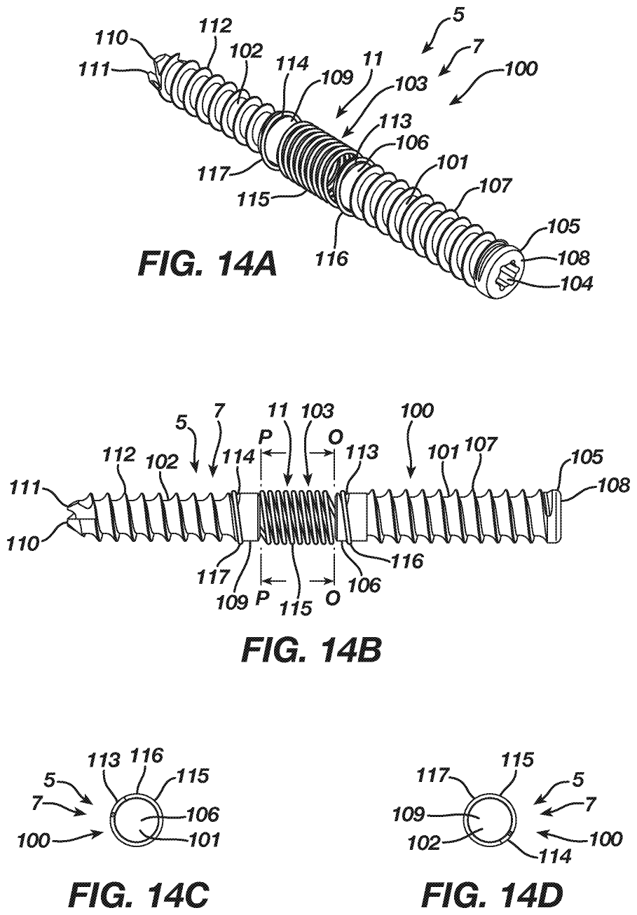

FIGS. 14A-14D illustrate the orthopedic fixation system 5 including the fastener 7 in the form of a screw 100 according to an eight embodiment. The screw 100 in the eighth embodiment includes an upper shaft 101, a lower shaft 102, an elastic member 103 providing the elastic component 11, a drive 104, and a second drive 16. The upper shaft 101 includes a top end 105, a bottom end 106, and a passage 19 from the top end 105 to the bottom end 106. The upper shaft 101 includes threads 107 about the upper shaft 101 or along at least a segment thereof. The upper shaft 101 at the top end 105 in the eighth embodiment includes a head 108 of the screw 100. The lower shaft 102 includes a top end 109, a bottom end 110 terminating in a point 111 of the screw 100, and a passage 25 from the top end 109 to the bottom end 110 and through the point 111. The lower shaft 102 includes threads 112 about the lower shaft 102 or along at least a segment thereof. The upper shaft 101 at the bottom end 106 and the lower shaft 102 at the top end 109 include respectively a groove 113 and a groove 114. The groove 113 at the bottom end 106 and the groove 114 at the top end 109 provide respectively an engagement point for the elastic member 103 with the upper shaft 101 and the lower shaft 102 such that the elastic member 103 connects the upper shaft 101 at the bottom end 106 thereof with the lower shaft 102 at the top end 109 thereof.

The drive 104 resides at the top end 105 of the upper shaft 101 and more particularly within the head 108 of the screw 100. The drive 104 in the eighth embodiment is any internal drive, such as the illustrated internal hexalobular drive often referred to as a star or torx drive, suitable for engagement by an external driver to facilitate a turning of the screw 100 at the upper shaft 101.

The elastic member 103 forming the elastic component 11 of the screw 100, which connects the upper shaft 101 at the bottom end 106 thereof with the lower shaft 102 at the top end 109 thereof, provides the screw 100 with the ability to accommodate relative motion between the first bone and the second bone. The elastic member 103 in the eighth embodiment includes a coil spring 115 comprised of a biocompatible shape memory material exhibiting superelastic properties (e.g., Nitinol) or elastic properties including but not limited to a metal or metal alloy, such as, for example, titanium, stainless steel, high carbon steel, titanium alloy, and cobalt chrome alloy.

The screw 100 including the elastic member 103 may be manufactured as follows. The upper and lower shafts 101 and 102 including the drive 104 and the grooves 113 and 114 of the screw 100 are formed from a biocompatible metal or metal alloy, such as, for example, titanium, stainless steel, titanium alloy, and cobalt chrome alloy, using suitable means, such as, for example, machining. After forming the upper and lower shafts 101 and 102, the coil spring 115 at a top coil 116 inserts over the upper shaft 101 at the bottom end 106 thereof and into the groove 113 thereby securing the coil spring 115 with the bottom end 106 of the upper shaft 101. Likewise, the coil spring 115 at a bottom coil 117 inserts over the lower shaft 102 at the top end 109 thereof and into the groove 114 thereby securing the coil spring 115 with the top end 109 of the lower shaft 102. The coil spring 115 in the eighth embodiment accordingly connects the upper shaft 101 at the bottom end 106 thereof with the lower shaft 102 at the top end 109 thereof while facilitating relative movement between the upper and lower shafts 101 and 102 of the screw 100.

The screw 100 as illustrated in FIGS. 14A-14D includes a natural insertion shape. Nevertheless, the screw 100 at the elastic member 103, due to the manufacture of the elastic member 103 in the form of the coil spring 115 from a shape memory material, is elastically deformable. In accordance therewith, the elastic member 103 and thus the screw 100 move from the natural insertion shape into an elastically deformed shape responsive to an applied force exerting stress on the screw 100. More particularly, the elastic member 103 and thus the screw 100 flex, elongate, and/or rotate during transition to the elastically deformed shape in response to an applied force exerting stress on the screw 100. While the elastic member 103 in the form of the coil spring 115 elastically deforms in response to an applied force exerting stress on the screw 100, the elastic member 103 in the eighth embodiment is sufficiently rigid to permit a rotation of the screw 100 during an insertion thereof into the first bone and the second bone whereby the screw 100 inserts into the first bone and the second bone in the natural insertion shape. If desirable to the operation of the screw 100, the rotation component of the elastic member 103 triggered by a stress on the screw 100 may be eliminated in order to ensure the screw 100 inserts into the first bone and the second bone in the natural insertion shape. The elastic member 103 and thus the screw 100 remain in the elastically deformed shape until removal of the applied force and the resulting stress exerted on the screw 100, whereupon, the elastic member 103 and thus the screw 100, due to the manufacture of the elastic member 103 in the form of the coil spring 115 from the shape memory material, returns from the elastically deformed shape to the natural insertion shape. The elastic member 103 in the form of the coil spring 115 and thus the screw 100 on account of the superelastic or elastic properties of the shape memory material is capable of repeated cycling between the natural insertion shape and an elastically deformed shape without experiencing a high cycle fatigue resulting in a breaking of the screw 100.

A bone repair involving the first bone and the second bone, such as, for example, a syndesmosis repair, includes using the screw 100 to affix the first bone and the second bone at, for example, the syndesmosis thereof. The screw 100, which resides in the natural insertion shape, inserts at the lower shaft 102 through the first bone and into the second bone and at the upper shaft 101 into the first bone. The lower shaft 102 inserts into the second bone and the upper shaft 101 inserts into the first bone until the elastic member 103 and more particularly the coil spring 115 resides between and thus spans the first bone and the second bone. In accordance with the lower shaft 102 and the upper shaft 101 inserting respectively into the second bone and the first bone with the elastic member 103 therebetween, the screw 100 fixates and thus stabilizes the first bone relative to the second bone while the elastic member 103 in the form of the coil spring 115 accommodates relative motion therebetween. During the healing process, natural patient movement invariably applies a force at the first bone and the second bone resulting in a relative motion therebetween that stresses the screw 100. Responsive to the stress thereon, the screw 100 at the elastic member 103 in the form of the coil spring 115, due to the superelastic or elastic properties of the shape memory material, moves, and, in particular, flexes, elongates, and/or rotates, during a transition from the natural insertion shape to the elastically deformed shape. The screw 100 at the elastic member 103 remains elastically deformed until a termination of the applied force at the first bone and the second bone and the resulting stress on the screw 100. The screw 100 at the elastic member 103, due to the superelastic or elastic properties of the shape memory material and in response to the termination of the stress thereon, returns from the elastically deformed shape to the natural insertion shape. Although natural patient movement during the healing process repeatedly stresses the screw 100, the screw 100 manages the recurring stress without breaking on the basis the elastic member 103 in the form of the coil spring 115 and thus the screw 100 on account of the superelastic or elastic properties of the shape memory material is capable of repeated cycling between the natural insertion shape and the elastically deformed shape without experiencing a high cycle fatigue resulting in a breaking of the screw 100. The screw 100 therefore provides an improvement in the bone repair of the first bone and the second bone because the screw 100 during the entire healing process fixates and stabilizes the first bone relative to the second bone while the screw 100 at the elastic member 103 in the form of the coil spring 115 accommodates relative motion therebetween, such as, for example, translational and rotational motion.

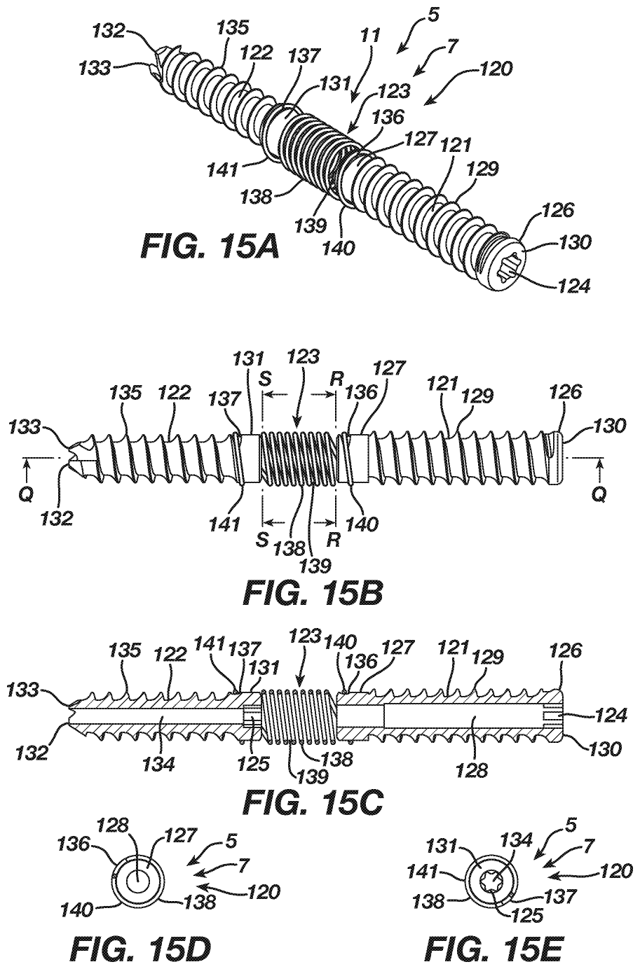

FIGS. 15A-15E illustrate the orthopedic fixation system 5 including the fastener 7 in the form of a screw 120 according to a ninth embodiment. The screw 120 in the ninth embodiment includes an upper shaft 121, a lower shaft 122, an elastic member 123 providing the elastic component 11, a first drive 124, and a second drive 125. The upper shaft 121 includes a top end 126, a bottom end 127, and a passage 128 from the top end 126 to the bottom end 127. The upper shaft 121 includes threads 129 about the upper shaft 121 or along at least a segment thereof. The upper shaft 121 at the top end 126 in the ninth embodiment includes a head 130 of the screw 120. The lower shaft 122 includes a top end 131, a bottom end 132 terminating in a point 133 of the screw 120, and a passage 134 from the top end 131 to the bottom end 132 and through the point 133. The lower shaft 122 includes threads 135 about the lower shaft 135 or along at least a segment thereof. The upper shaft 121 at the bottom end 127 and the lower shaft 122 at the top end 131 include respectively a groove 136 and a groove 137. The groove 136 at the bottom end 127 and the groove 137 at the top end 131 provide respectively an engagement point for the elastic member 123 with the upper shaft 121 and the lower shaft 122 such that the elastic member 123 connects the upper shaft 121 at the bottom end 127 thereof with the lower shaft 122 at the top end 131 thereof.

The first drive 124 resides at the top end 126 of the upper shaft 121 and more particularly within the head 130 of the screw 120. The first drive 124 in the ninth embodiment is any internal drive, such as the illustrated internal hexalobular drive often referred to as a star or torx drive, suitable for engagement by an external driver to facilitate a turning of the screw 120 at the upper shaft 121. The first drive 124 communicates with the passage 128 to allow an external driver as will be described more fully herein to pass through the upper shaft 121 and engage the second drive 125. The first drive 124 and the passage 128 further allow the screw 120 at the upper shaft 121 to insert over a guide wire utilized to hold the first bone and the second bone during insertion of the screw 120.

The second drive 125 resides at the top end 131 of the lower shaft 122. The second drive 125 in the ninth embodiment is any internal drive, such as the illustrated internal hexalobular drive often referred to as a star or torx drive, suitable for engagement by an external driver to facilitate a turning of the screw 120 at the lower shaft 122. The second drive 125 communicates with the passage 134 to allow the screw 120 at the lower shaft 122 to insert over a guide wire utilized to hold the first bone and the second bone during insertion of the screw 120.

The elastic member 123 forming the elastic component 11 of the screw 120, which connects the upper shaft 121 at the bottom end 127 thereof with the lower shaft 122 at the top end 131 thereof, provides the screw 120 with the ability to accommodate relative motion between the first bone and the second bone. The elastic member 123 in the ninth embodiment includes a coil spring 138 comprised of a biocompatible shape memory material exhibiting superelastic properties (e.g., Nitinol) or elastic properties including but not limited to a metal or metal alloy, such as, for example, titanium, stainless steel, high carbon steel, titanium alloy, and cobalt chrome alloy. The elastic member 123, due to the coil spring 138, includes a passage 139 therethrough. The passage 139 communicates with the passage 128 of the upper shaft 121 at the bottom end 127 thereof while being open to the second drive 125 at the top end 131 of the lower shaft 122 to allow an external driver to pass through the upper shaft 121 and the elastic member 123 and engage the second drive 125. The passage 139 allows the screw 120 to insert over a guide wire utilized to hold the first bone and the second bone during insertion of the screw 120.

The screw 120 including the elastic member 123 may be manufactured as follows. The upper and lower shafts 121 and 122 including the first and second drives 124 and 125 and the grooves 136 and 137 of the screw 120 are formed from a biocompatible metal or metal alloy, such as, for example, titanium, stainless steel, titanium alloy, and cobalt chrome alloy, using suitable means, such as, for example, machining. After forming the upper and lower shafts 121 and 122, the coil spring 138 at a top coil 140 inserts over the upper shaft 121 at the bottom end 127 thereof and into the groove 136 thereby securing the coil spring 138 with the bottom end 127 of the upper shaft 121. Likewise, the coil spring 138 at a bottom coil 141 inserts over the lower shaft 122 at the top end 131 thereof and into the groove 137 thereby securing the coil spring 138 with the top end 131 of the lower shaft 122. The coil spring 138 in the ninth embodiment accordingly connects the upper shaft 121 at the bottom end 127 thereof with the lower shaft 122 at the top end 131 thereof while facilitating relative movement between the upper and lower shafts 121 and 122 of the screw 120.

The screw 120 as illustrated in FIGS. 15A-15D includes a natural insertion shape. Nevertheless, the screw 120 at the elastic member 123, due to the manufacture of the elastic member 123 in the form of the coil spring 138 from a shape memory material, is elastically deformable. In accordance therewith, the elastic member 123 and thus the screw 120 move from the natural insertion shape into an elastically deformed shape responsive to an applied force exerting stress on the screw 120. More particularly, the elastic member 123 and thus the screw 120 flex, elongate, and/or rotate during transition to the elastically deformed shape in response to an applied force exerting stress on the screw 120. The elastic member 123 and thus the screw 120 remain in the elastically deformed shape until removal of the applied force and the resulting stress exerted on the screw 120, whereupon, the elastic member 123 and thus the screw 120, due to the manufacture of the elastic member 123 in the form of the coil spring 138 from the shape memory material, returns from the elastically deformed shape to the natural insertion shape. The elastic member 123 in the form of the coil spring 138 and thus the screw 120 on account of the superelastic or elastic properties of the shape memory material is capable of repeated cycling between the natural insertion shape and an elastically deformed shape without experiencing a high cycle fatigue resulting in a breaking of the screw 120.

A bone repair involving the first bone and the second bone, such as, for example, a syndesmosis repair, includes using the screw 120 to affix the first bone and the second bone at, for example, the syndesmosis thereof. The screw 120, which resides in the natural insertion shape, inserts at the lower shaft 122 through the first bone and into the second bone and at the upper shaft 121 into the first bone. The lower shaft 122 inserts into the second bone and the upper shaft 121 inserts into the first bone until the elastic member 123 and more particularly the coil spring 138 resides between and thus spans the first bone and the second bone. In accordance with the lower shaft 122 and the upper shaft 121 inserting respectively into the second bone and the first bone with the elastic member 123 therebetween, the screw 120 fixates and thus stabilizes the first bone relative to the second bone while the elastic member 123 in the form of the coil spring 138 accommodates relative motion therebetween. During the healing process, natural patient movement invariably applies a force at the first bone and the second bone resulting in a relative motion therebetween that stresses the screw 120. Responsive to the stress thereon, the screw 120 at the elastic member 123 in the form of the coil spring 138, due to the superelastic or elastic properties of the shape memory material, moves, and, in particular, flexes, elongates, and/or rotates, during a transition from the natural insertion shape to the elastically deformed shape. The screw 120 at the elastic member 123 remains elastically deformed until a termination of the applied force at the first bone and the second bone and the resulting stress on the screw 120. The screw 120 at the elastic member 123, due to the superelastic or elastic properties of the shape memory material and in response to the termination of the stress thereon, returns from the elastically deformed shape to the natural insertion shape. Although natural patient movement during the healing process repeatedly stresses the screw 120, the screw 120 manages the recurring stress without breaking on the basis the elastic member 123 in the form of the coil spring 138 and thus the screw 120 on account of the superelastic or elastic properties of the shape memory material is capable of repeated cycling between the natural insertion shape and the elastically deformed shape without experiencing a high cycle fatigue resulting in a breaking of the screw 120. The screw 120 therefore provides an improvement in the bone repair of the first bone and the second bone because the screw 120 during the entire healing process fixates and stabilizes the first bone relative to the second bone while the screw 120 at the elastic member 123 in the form of the coil spring 138 accommodates relative motion therebetween, such as, for example, translational and rotational motion.

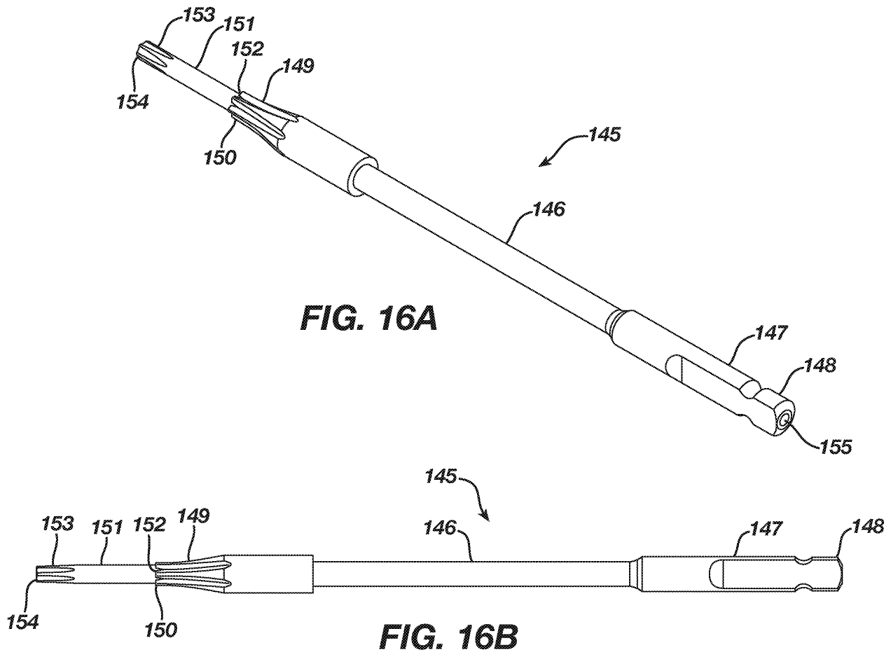

FIGS. 16A-16B illustrate a driver instrument 145 utilized in inserting the screws 10, 30, 35, and 120 according to the first, second, third, and ninth embodiments at the lower shafts 13 and 122 thereof through the first bone and into the second bone and at the upper shafts 12 and 121 thereof into the first bone. The driver instrument 145 includes an upper shaft 146 having a shank 147 at a top end 148 and a first driver 149 at a bottom end 150. The shank 147 is configured to facilitate engagement of the driver instrument 145 by a suitable tool such as a handle, wrench, or drill used to rotate the driver instrument 145 during insertion of the screws 10, 30, 35, and 120. The driver instrument 145 includes a lower shaft 151 extending at a top end 152 from the bottom end 150 of the upper shaft 146 and terminating in a second driver 153 at a bottom end 154. The driver instrument 145 preferably is cannulated whereby a passage 155 traverses the driver instrument 145 from the top end 148 of the upper shaft 146 to the bottom end 154 of the lower shaft 151. The passage 155 allows the driver instrument 145 to insert over a guide wire utilized to hold the first bone and the second bone during insertion of one or more of the screws 10, 30, 35, and 120.

The first driver 149 is any external drive, such as the illustrated external hexalobular driver often referred to as a star or torx driver, suitable for engagement with one of the first drives 15 and 124 to facilitate a turning of one of the screws 10, 30, 35, and 120 at the upper shafts 12 and 121. Similarly, the second driver 153 is any external drive, such as the illustrated external hexalobular drive often referred to as a star or torx drive, suitable for engagement with one of the second drives 16 and 125 to facilitate a turning of one of the screw 10, 30, 35, and 120 at the lower shafts 13 and 122.

The driver instrument 145 in use inserts at the lower shaft 151 into one of the passages 19 and 128 of the upper shafts 12 and 121 and then into one of the passages 28 and 139 of the elastic members 14 and 123 until the second driver 153 seats within one of the second drives 16 and 125. Concurrent with the seating of the second driver 153 within one of the second drives 16 and 125, the first driver 149 seats within one of the first drives 15 and 124. The first driver 149 seats within one of the first drives 15 and 124 concurrent with the seating of the second driver 153 seats within one of the second drives 16 and 125 on the basis the distances between the first drives 15 and 124 and the second drives 16 and 125 and the first driver 149 and the second driver 153 are the same. The seating of the first driver 149 within one of the first drives 15 and 124 and the second driver 153 within one of the second drives 16 and 125 allows insertion of one of the screws 10, 30, 35, and 120 at the lower shafts 13 and 122 thereof through the first bone and into the second bone and at the upper shafts 12 and 121 thereof into the first bone. More particularly, a rotation of the driver instrument 145 at the shank 147 causes the first driver 149 at one of the first drives 15 and 124 and the second driver 153 at one of the second drives 16 and 125 to turn one of the screws 10, 30, 35, and 120 respectively at the upper shafts 12 and 121 thereof and the lower shafts 13 and 122 thereof. In accordance therewith, one of the lower shafts 13 and 122 inserts through the first bone and into the second bone while one of the upper shafts 12 and 121 inserts into the first bone. The driver instrument 145 additionally, due to the engagements of one of the first drives 15 and 124 and second drives 16 and 125 respectively by the first and second drivers 149 and 153 and the resulting rigid holding of the upper shafts 12 and 121 relative to the lower shafts 13 and 122, maintains the screws 10, 30, 35, and 120 in the natural insertion shape during implantation thereof within the first and second bones.

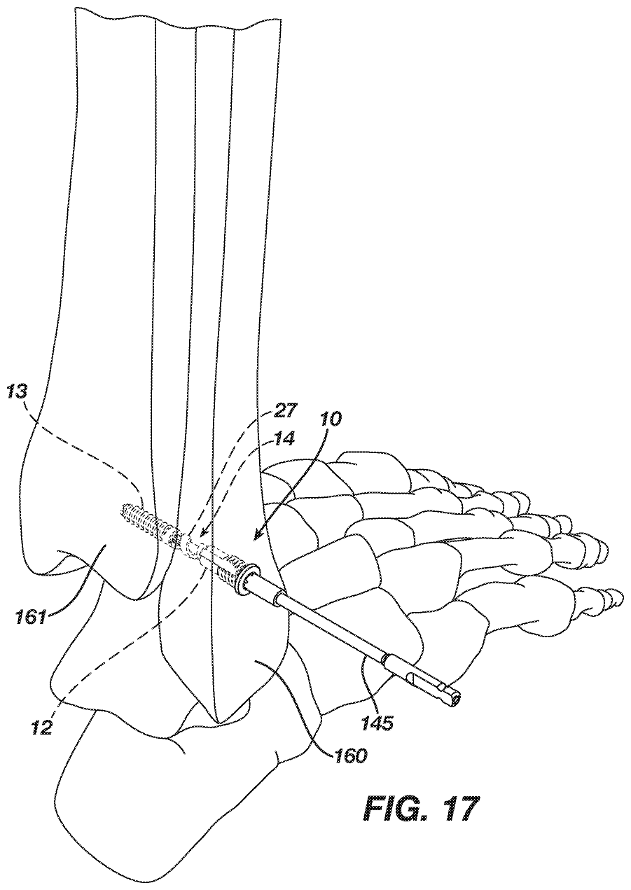

FIG. 17 illustrates use of a screw 10 according to the first embodiment in an example bone repair involving a first bone 160 and a second bone 161, such as, for example, a syndesmosis repair between the first bone 160 and the second bone 161. With the screw 10 loaded on a driver instrument 145 whereby the driver instrument 145 retains the screw 10 in the natural insertion shape, the first bone 160 aligns with the second bone 161 in an orientation that promotes fixation of the first bone 160 with the second bone 161 and a proper syndesmosis repair. If desired pilot holes for the screw 10 may be drilled in the first and second bones 160 and 161. The screw 10 using the driver instrument 145 inserts at the lower shaft 13 through the first bone 160 and into the second bone 161 and at the upper shaft 12 into the first bone 160 whereby the screw 10 affixes the first bone 160 and the second bone 161 at, for example, the syndesmosis thereof. The lower shaft 13 inserts into the second bone 161 and the upper shaft 12 inserts into the first bone 160 until the elastic member 14 and more particularly the helical spring 27 resides between and thus spans the first bone 160 and the second bone 161. In accordance with the lower shaft 13 and the upper shaft 12 inserting respectively into the second bone 161 and the first bone 160 with the elastic member 14 therebetween, the screw 10 fixates and thus stabilizes the first bone 160 relative to the second bone 161 while the elastic member 14 in the form of the helical spring 27 accommodates relative motion therebetween. During the healing process, natural patient movement invariably applies a force at the first bone 160 and the second bone 161 resulting in a relative motion therebetween that stresses the screw 10. Responsive to the stress thereon, the screw 10 at the elastic member 14 in the form of the helical spring 27, due to the superelastic properties of the shape memory material, moves, and, in particular, flexes, elongates, and/or rotates, during a transition from the natural insertion shape to the elastically deformed shape. The screw 10 at the elastic member 14 remains elastically deformed until a termination of the applied force at the first bone 160 and the second bone 161 and the resulting stress on the screw 10. The screw 10 at the elastic member 14, due to the superelastic properties of the shape memory material and in response to the termination of the stress thereon, returns from the elastically deformed shape to the natural insertion shape. Although natural patient movement during the healing process repeatedly stresses the screw 10, the screw 10 manages the recurring stress without breaking on the basis the elastic member 14 in the form of the helical spring 27 and thus the screw 10 on account of the superelastic properties of the shape memory material is capable of repeated cycling between the natural insertion shape and the elastically deformed shape without experiencing a high cycle fatigue resulting in a breaking of the screw 10. The screw 10 therefore provides an improvement in the bone repair of the first bone 160 and the second bone 161 because the screw 10 during the entire healing process fixates and stabilizes the first bone 160 relative to the second bone 161 while the screw 10 at the elastic member 14 in the form of the helical spring 27 accommodates relative motion therebetween, such as, for example, translational and rotational motion.

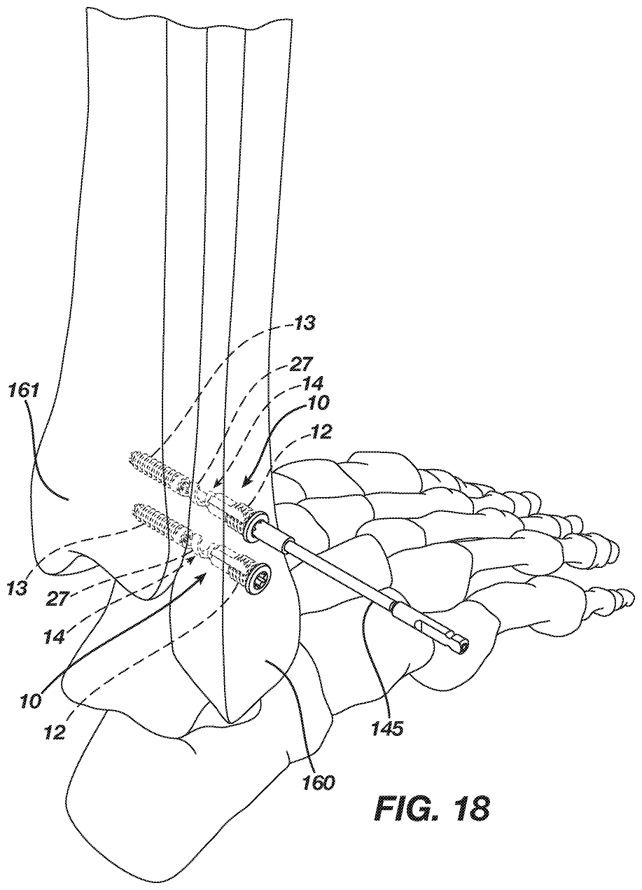

FIG. 18 illustrates use of a second screw 10 according to the first embodiment in the example bone repair involving the first bone 160 and the second bone 161. The second screw 10 inserts at a second insertion point within the first bone 160 and the second bone 161 the same as the previous screw 10 in order to provide additional fixation between the first bone 160 and the second bone 161 while still accommodating relative motion therebetween.

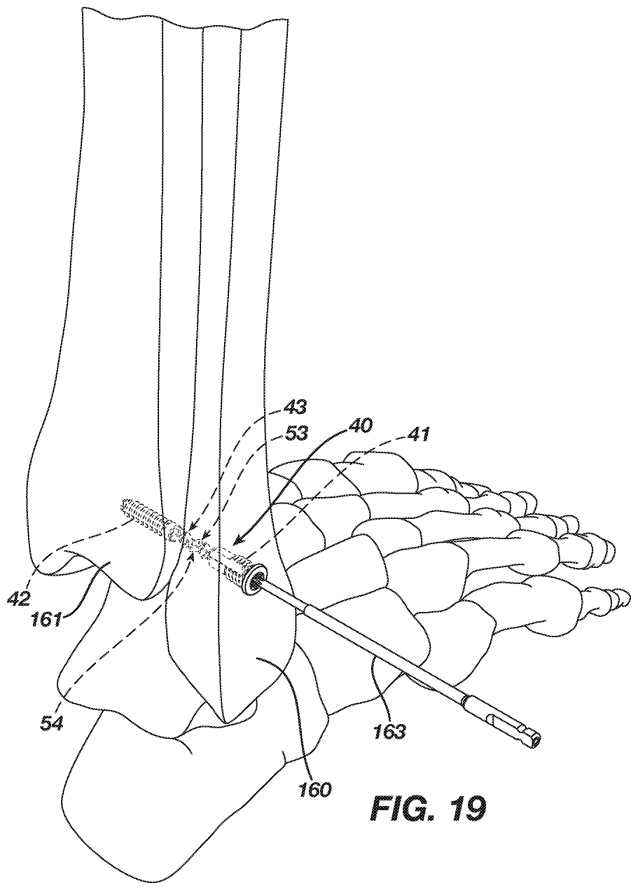

FIG. 19 illustrates use of a screw 40 according to the fourth embodiment in an example bone repair involving the first bone 160 and the second bone 161, such as, for example, a syndesmosis repair between the first bone 160 and the second bone 161. With the screw 40 loaded on a single stage driver instrument 163, the first bone 160 aligns with the second bone 161 in an orientation that promotes fixation of the first bone 160 with the second bone 161 and a proper syndesmosis repair. If desired pilot holes for the screw 40 may be drilled in the first and second bones 160 and 161. The screw 40 using the driver instrument 163 inserts at the lower shaft 42 through the first bone 160 and into the second bone 161 and at the upper shaft 41 into the first bone 160 whereby the screw 40 affixes the first bone 160 and the second bone 161 at, for example, the syndesmosis thereof. The lower shaft 42 inserts into the second bone 161 and the upper shaft 41 inserts into the first bone 160 until the elastic member 43 and more particularly the series 53 of interconnected individual geometric shapes 54 resides between and thus spans the first bone 160 and the second bone 161. In accordance with the lower shaft 42 and the upper shaft 41 inserting respectively into the second bone 161 and the first bone 160 with the elastic member 43 therebetween, the screw 40 fixates and thus stabilizes the first bone 160 relative to the second bone 161 while the elastic member 43 in the form of the series 53 of interconnected individual geometric shapes 54 accommodates relative motion therebetween. During the healing process, natural patient movement invariably applies a force at the first bone 160 and the second bone 161 resulting in a relative motion therebetween that stresses the screw 40. Responsive to the stress thereon, the screw 40 at the elastic member 43 in the form of the series 53 of interconnected individual geometric shapes 54, due to the superelastic properties of the shape memory material, moves, and, in particular, flexes, elongates, and/or rotates, during a transition from the natural insertion shape to the elastically deformed shape. The screw 40 at the elastic member 43 remains elastically deformed until a termination of the applied force at the first bone 160 and the second bone 160 and the resulting stress on the screw 40. The screw 40 at the elastic member 43, due to the superelastic properties of the shape memory material and in response to the termination of the stress thereon, returns from the elastically deformed shape to the natural insertion shape. Although natural patient movement during the healing process repeatedly stresses the screw 40, the screw 40 manages the recurring stress without breaking on the basis the elastic member 43 in the form of the series 53 of interconnected individual geometric shapes 54 and thus the screw 40 on account of the superelastic properties of the shape memory material is capable of repeated cycling between the natural insertion shape and the elastically deformed shape without experiencing a high cycle fatigue resulting in a breaking of the screw 40. The screw 40 therefore provides an improvement in the bone repair of the first bone 160 and the second bone 161 because the screw 40 during the entire healing process fixates and stabilizes the first bone 160 relative to the second bone 161 while the screw 40 at the elastic member 43 in the form of the series 53 of interconnected individual geometric shapes 54 accommodates relative motion therebetween, such as, for example, translational and rotational motion.

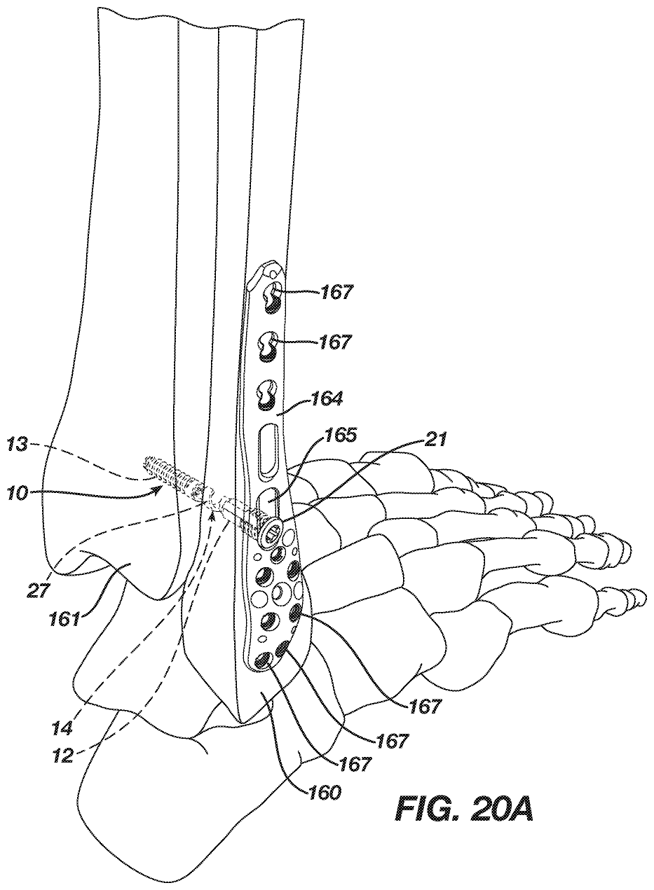

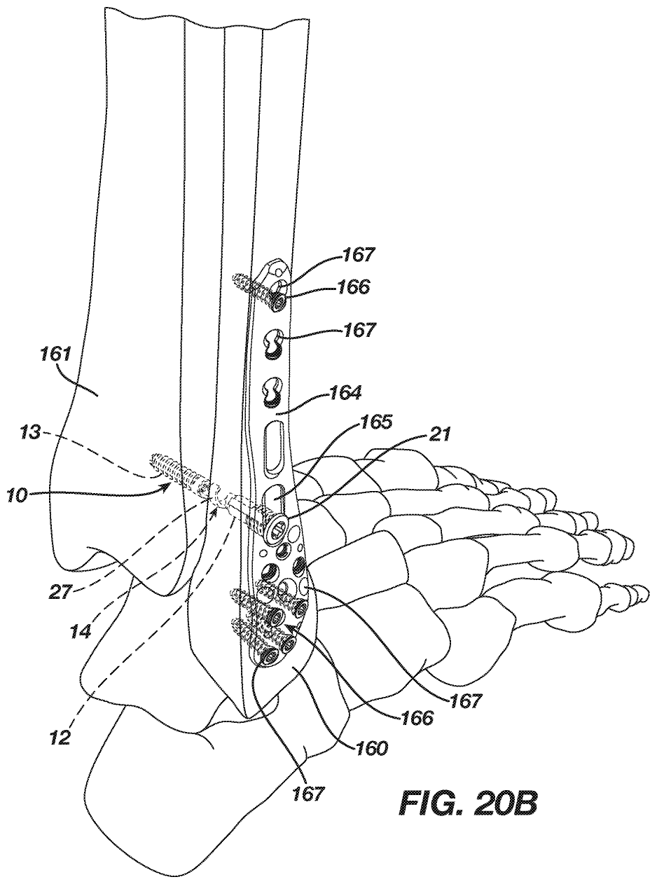

FIGS. 20A-20B illustrate use of a screw 10 according to the first embodiment and a bone plate 164 in an example bone repair involving the first bone 160 and the second bone 161, such as, for example, a syndesmosis repair between the first bone 160 and the second bone 161. With the screw 10 loaded on the driver instrument 145 whereby the driver instrument 145 retains the screw 10 in the natural insertion shape, the first bone 160 aligns with the second bone 161 in an orientation that promotes fixation of the first bone 160 with the second bone 161 and a proper syndesmosis repair. The bone plate 164 seats atop the first bone 160 with a screw hole 165 thereof located at a desired insertion point for the screw 10 into the first and second bones 160 and 161. If desired pilot holes for the screw 10 may be drilled in the first and second bones 160 and 161. The screw 10 using the driver instrument 145 inserts at the lower shaft 13 through the screw hole 165 and the first bone 160 and into the second bone 161 and at the upper shaft 12 through the screw hole 165 and into the first bone 160 whereby the screw 10 affixes the first bone 160 and the second bone 161 at, for example, the syndesmosis thereof. The lower shaft 13 inserts into the second bone 161 and the upper shaft 12 inserts into the first bone 160 until the elastic member 14 and more particularly the helical spring 27 resides between and thus spans the first bone 160 and the second bone 161 and the screw 10 at the head 21 engages the bone plate 165 thereby securing the bone plate 164 to the first bone 160. In the event additional fixation of the bone plate 165 to the first bone 160 is desired, one or more screws 166 may be inserted through one or more screw holes 167 of the bone plate 165 and into the first bone 160. In accordance with the lower shaft 13 and the upper shaft 12 inserting respectively into the second bone 161 and the first bone 160 with the elastic member 14 therebetween and the fixation of the bone plate 165 to the first bone 160, the screw 10 fixates and thus stabilizes the first bone 160 relative to the second bone 161 while the elastic member 14 in the form of the helical spring 27 accommodates relative motion therebetween. During the healing process, natural patient movement invariably applies a force at the first bone 160 and the second bone 161 resulting in a relative motion therebetween that stresses the screw 10. Responsive to the stress thereon, the screw 10 at the elastic member 14 in the form of the helical spring 27, due to the superelastic properties of the shape memory material, moves, and, in particular, flexes, elongates, and/or rotates, during a transition from the natural insertion shape to the elastically deformed shape. The screw 10 at the elastic member 14 remains elastically deformed until a termination of the applied force at the first bone 160 and the second bone 161 and the resulting stress on the screw 10. The screw 10 at the elastic member 14, due to the superelastic properties of the shape memory material and in response to the termination of the stress thereon, returns from the elastically deformed shape to the natural insertion shape. Although natural patient movement during the healing process repeatedly stresses the screw 10, the screw 10 manages the recurring stress without breaking on the basis the elastic member 14 in the form of the helical spring 27 and thus the screw 10 on account of the superelastic properties of the shape memory material is capable of repeated cycling between the natural insertion shape and the elastically deformed shape without experiencing a high cycle fatigue resulting in a breaking of the screw 10. The screw 10 therefore provides an improvement in the bone repair of the first bone 160 and the second bone 161 because the screw 10 during the entire healing process fixates and stabilizes the first bone 160 relative to the second bone 161 while the screw 10 at the elastic member 14 in the form of the helical spring 27 accommodates relative motion therebetween, such as, for example, translational and rotational motion.

Although the present invention has been described in terms of the foregoing preferred embodiments, such description has been for exemplary purposes only and, as will be apparent to those of ordinary skill in the art, many alternatives, equivalents, and variations of varying degrees will fall within the scope of the present invention. That scope, accordingly, is not to be limited in any respect by the foregoing detailed description; rather, it is defined only by the claims that follow.

1-15. (canceled)

16. An orthopedic fixation system, comprising:
a screw including a natural insertion shape, the screw being configured to secure a first bone with a second bone whereby the screw stabilizes the first bone relative to the second bone, the screw, comprising:
an upper shaft with a top end and a bottom end, the upper shaft including threads about at least a segment thereof,
a lower shaft with a top end and a bottom end, the lower shaft including threads about at least a segment thereof, and
a series of interconnected individual geometric shapes formed from a superelastic shape memory material connecting the upper shaft at the bottom end thereof with the lower shaft at the top end thereof;
the series of interconnected individual geometric shapes being configured to allow the screw to move from the natural insertion shape to an elastically deformed shape in response to a stress applied to the screw during a relative motion between the first bone and the second bone;
the series of interconnected individual geometric shapes being configured to allow the screw to return from the elastically deformed shape to the natural insertion shape upon a removal of the stress applied to the screw; and
the series of interconnected individual geometric shapes being configured to provide the screw with the ability to repeatedly cycle between the natural insertion shape and the elastically deformed shape such that the screw accommodates relative motion between the first bone relative to the second bone.

17. The orthopedic fixation system of claim 16, wherein the screw in the natural insertion shape being configured to insert at the lower shaft through the first bone and into the second bone and at the upper shaft into the first bone, whereby the lower shaft inserts into the second bone and the upper shaft inserts into the first bone until the series of interconnected individual geometric shapes resides between the first bone and the second bone.

18. An orthopedic fixation system, comprising:
a screw including a natural insertion shape, the screw being configured to secure a first bone with a second bone whereby the screw stabilizes the first bone relative to the second bone, the screw, comprising:
an upper shaft with a top end and a bottom end, the upper shaft including threads about at least a segment thereof,
a lower shaft with a top end and a bottom end, the lower shaft including threads about at least a segment thereof, and
an elastomer connecting the upper shaft at the bottom end thereof with the lower shaft at the top end thereof;
the elastomer being configured to allow the screw to move from the natural insertion shape to an elastically deformed shape in response to a stress applied to the screw during a relative motion between the first bone and the second bone;
the elastomer configured to allow the screw to return from the elastically deformed shape to the natural insertion shape upon a removal of the stress applied to the screw; and
the elastomer being configured to provide the screw with the ability to repeatedly cycle between the natural insertion shape and the elastically deformed shape such that the screw accommodates relative motion between the first bone relative to the second bone.

19. The orthopedic fixation system of claim 18, wherein:
the upper shaft includes a bore from the bottom end to within the upper shaft; and
the lower shaft includes a bore from the top end to within the lower shaft.

20. The orthopedic fixation system of claim 19, wherein the elastomer forms a bridge with an upper rod extending from the bridge at the top end thereof to within the bore of the upper shaft and a lower rod extending from the bridge at the bottom end thereof to within the bore of the lower shaft such that the bridge at the upper rod and the lower rod connects the upper shaft at the bottom end thereof with the lower shaft at the top end thereof while residing therebetween.

21. The orthopedic fixation system of claim 20, wherein the screw in the natural insertion shape being configured to insert at the lower shaft through the first bone and into the second bone and at the upper shaft into the first bone, whereby the lower shaft inserts into the second bone and the upper shaft inserts into the first bone until the bridge resides between the first bone and the second bone.

22. The orthopedic fixation system of claim 18, wherein:
the upper shaft includes at least one fin extending from the bottom end thereof;
the lower shaft includes at least one cavity from the top end to within the lower shaft; and
the fin and the cavity being configured whereby a positioning of the upper shaft at the bottom end adjacent the lower shaft at the top end and an insertion of the fin into the cavity creates a channel running between the bottom end of the upper shaft and the top end of the lower shaft and the fin and the cavity.

23. The orthopedic fixation system of claim 22, wherein the elastomer comprises an elastomer filling disposed within the channel to form a bridge between the bottom end of the upper shaft and the top end of the lower shaft and the fin and the cavity such that the bridge connects the upper shaft at the bottom end thereof with the lower shaft at the top end thereof.

24. The orthopedic fixation system of claim 23, wherein the screw in the natural insertion shape being configured to insert at the lower shaft through the first bone and into the second bone and at the upper shaft into the first bone, whereby the lower shaft inserts into the second bone and the upper shaft inserts into the first bone until the bridge resides between the first bone and the second bone.
